(19) United States
(12) Patent Application Publication
(10) Pub. No.: US 2017/0319552 A1
(43) Pub. Date: Nov. 9, 2017

(54) ATROPISOMERS OF TRIAZOLE DERIVATIVE

(71) Applicant: ARDEA BIOSCIENCES, INC., San Diego, CA (US)

(72) Inventor: Matthew Kent RENNER, San Diego, CA (US)

(21) Appl. No.: 15/440,722

(22) Filed: Feb. 23, 2017

Related U.S. Application Data
(60) Provisional application No. 62/299,509, filed on Feb. 24, 2016.

(51) Int. Cl. A61K 31/4196; C07D 249/12; A61K 31/03

(52) U.S. Cl. CPC A61K 31/4196; A61K 31/03; C07D 249/12

(57) ABSTRACT

Atropisomers of 2-(5-bromo-4-(4-cyclopropyl naphthalen-1-yl)-4H- 1,2,4-triazol-3-ylthio)acetic acid are described. Pharmaceutical compositions and the uses of such compounds, compound forms, and compositions for the treatment of a variety of diseases and conditions are also presented.

### CROSS-REFERENCE TO RELATED APPLICATIONS

This application claims the benefit of U.S. Provisional Application Ser. No. 62/299,509 filed Feb. 24, 2016, which is hereby incorporated by reference in its entirety.

### BACKGROUND OF THE INVENTION

Gout is associated with elevated levels of uric acid that crystallize and deposit in joints, tendons, and surrounding tissues. Gout is marked by recurrent attacks of red, tender, hot, and/or swollen joints.

### SUMMARY OF THE INVENTION

In one aspect provided herein is (+)-2-(5-bromo-4-(4-cyclopropylnaphthalen-1-yl)-4H-1,2,4-triazol-3-yl)thio)acetic acid.

In another aspect provided herein is (−)-2-((5-bromo-4-(4-cyclopropylnaphthalen-1-yl)-4H-1,2,4-triazol-3-yl)thio)acetic acid.

In another aspect provided herein is an atropisomer of 2-((5-bromo-4-(4-cyclopropylnaphthalen-1-yl)-4H-1,2,4-triazol-3-yl)thio)acetic acid, or a pharmaceutically acceptable salt thereof, wherein the atropisomer is (P)-2-((5-bromo-4-(4-cyclopropylnaphthalen-1-yl)-4H-1,2,4-triazol-3-yl)thio)acetic acid:

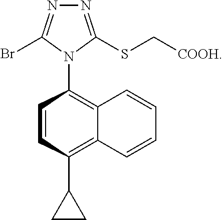

In yet another aspect provided herein is an atropisomer of 2-((5-bromo-4-(4-cyclopropylnaphthalen-1-yl)-4H-1,2,4-triazol-3-yl)thio)acetic acid, or a pharmaceutically acceptable salt thereof, wherein the atropisomer is (M)-2-((5-bromo-4-(4-cyclopropylnaphthalen-1-yl)-4H-1,2,4-triazol-3-yl)thio)acetic acid:

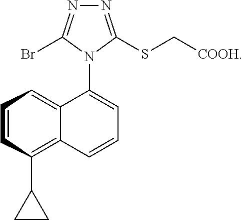

In some aspect, provided herein is a single atropisomer of 2-(5-bromo-4-(4-cyclopropylnaphthalen-1-yl)-4H-1,2,4-triazol-3-yl)thio)acetic acid), or a pharmaceutically acceptable salt thereof, wherein the atropisomer shows increased URAT-1 inhibition over the other atropisomer. In some embodiments, the single atropisomer is (+)-2-((5-bromo-4-(4-cyclopropylnaphthalen-1-yl)-4H-1,2,4-triazol-3-yl)thio)acetic acid).

In another aspect, provided herein is a pharmaceutical composition comprising either:
(i) (+)-2-((5-bromo-4-(4-cyclopropylnaphthalen-1-yl)-4H-1,2,4-triazol-3-yl)thio)acetic acid, or a pharmaceutically acceptable salt thereof; or
(ii) (−)-2-((5-bromo-4-(4-cyclopropylnaphthalen-1-yl)-4H-1,2,4-triazol-3-yl)thio)acetic acid, or a pharmaceutically acceptable salt thereof; or
(iii) a mixture enriched in one atropisomer of 2-((5-bromo-4-(4-cyclopropylnaphthalen-1-yl)-4H-1,2,4-triazol-3-yl)thio)acetic acid; or a pharmaceutically acceptable salt thereof; and a pharmacologically acceptable carrier, diluent, or excipient.

In some embodiments, the pharmaceutical composition further comprises:
(i) allopurinol; or
(ii) febuxostat; or
(iii) colchicine; or
(iv) any combination thereof.

In another aspect, provided herein is a method of inhibiting URAT-1, comprising contacting the URAT-1 receptor with an effective inhibiting amount of:
(i) (+)-2-((5-bromo-4-(4-cyclopropylnaphthalen-1-yl)-4H-1,2,4-triazol-3-yl)thio)acetic acid, or a pharmaceutically acceptable salt thereof; or
(ii) (−)-2-((5-bromo-4-(4-cyclopropylnaphthalen-1-yl)-4H-1,2,4-triazol-3-yl)thio)acetic acid, or a pharmaceutically acceptable salt thereof; or
(iii) a mixture enriched in one atropisomer of 2-((5-bromo-4-(4-cyclopropylnaphthalen-1-yl)-4H-1,2,4-triazol-3-yl)thio)acetic acid, or a pharmaceutically acceptable salt thereof.

In another aspect, provided herein is a method of reducing serum uric acid levels in a human, comprising administering to the human a therapeutically effective amount of:
(i) (+)-2-((5-bromo-4-(4-cyclopropylnaphthalen-1-yl)-4H-1,2,4-triazol-3-yl)thio)acetic acid, or a pharmaceutically acceptable salt thereof; or
(ii) (−)-2-((5-bromo-4-(4-cyclopropylnaphthalen-1-yl)-4H-1,2,4-triazol-3-yl)thio)acetic acid, or a pharmaceutically acceptable salt thereof; or
(iii) a mixture enriched in one atropisomer of 2-((5-bromo-4-(4-cyclopropylnaphthalen-1-yl)-4H-1,2,4-triazol-3-yl)thio)acetic acid, or a pharmaceutically acceptable salt thereof.

In another aspect, provided herein is a method of:
a. treating hyperuricemia in a human; or
b. treating gout in a human; or
c. treating hyperuricemia in a human with gout; or
d. preventing hyperuricemia in a human; or
e. preventing gout in a human; or
f achieving a therapeutic benefit in a human with gout; or
g. a combination thereof;
comprising administering to the human a therapeutically effective amount of:
   (i) (+)-2-((5-bromo-4-(4-cyclopropylnaphthalen-1-yl)-4H-1,2,4-triazol-3-yl)thio)acetic acid, or a pharmaceutically acceptable salt thereof; or
   (ii) (−)-2-((5-bromo-4-(4-cyclopropylnaphthalen-1-yl)-4H-1,2,4-triazol-3-yl)thio)acetic acid, or a pharmaceutically acceptable salt thereof; or
   (iii) a mixture enriched in one atropisomer of 2-((5-bromo-4-(4-cyclopropylnaphthalen-1-yl)-4H-1,2,4-triazol-3-yl)thio)acetic acid, or a pharmaceutically acceptable salt thereof.

In some embodiments the methods comprise administering to the human a therapeutically effective amount of (+)-2-((5-bromo-4-(4-cyclopropylnaphthalen-1-yl)-4H-1,2,4-triazol-3-yl)thio)acetic acid, or a pharmaceutically acceptable salt thereof.

In some embodiments the methods comprise administering to the human a therapeutically effective amount of mixture enriched in (+)-2-((5-bromo-4-(4-cyclopropylnaphthalen-1-yl)-4H-1,2,4-triazol-3-yl)thio)acetic acid, or a pharmaceutically acceptable salt thereof.

In some embodiments the methods comprise administering to the human a therapeutically effective amount of (−)-2-((5-bromo-4-(4-cyclopropylnaphthalen-1-yl)-4H-1,2,4-triazol-3-yl)thio)acetic acid, or a pharmaceutically acceptable salt thereof.

In some embodiments the methods comprise administering to the human a therapeutically effective amount of mixture enriched in (−)-2-((5-bromo-4-(4-cyclopropylnaphthalen-1-yl)-4H-1,2,4-triazol-3-yl)thio)acetic acid, or a pharmaceutically acceptable salt thereof.

In some embodiments the methods further comprise administering:

(i) allopurinol; or

(ii) febuxostat; or

(iii) colchicine; or

(iv) any combination thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1B show chromatograms of a Compound (I) Standard Solution (FIG. 1A full scale and FIG. 1B expanded scale).

FIG. 2A-FIG. 2B show chromatograms of atropisomer 1 in acetonitrile (FIG. 2A at t₀ control and FIG. 2B at t=4 Days at 60° C.).

FIG. 3A-FIG. 3C show chromatograms of atropisomer 1 in aqueous solution (reconstituted) (FIG. 3A at t₀ control, FIG. 3B at t=4 Days at 60° C., and FIG. 3C at t=4 Days at 100° C.).

FIG. 4A-FIG. 4D show graphs showing the formation of metabolites for atropisomer 1 and atropisomer 2 in Human Liver Microsomes (FIG. 4A M3c metabolite, FIG. 4B M3 metabolite, FIG. 4C M4 metabolite, and FIG. 4D M6 metabolite).

FIG. 5A-FIG. 5B show graphs showing the formation of metabolites for atropisomer 1 and atropisomer 2 in Human Liver Microsomes in the presence or absence of mEH Inhibitor Valpromide (VPM, 100 μM) (FIG. 5A M3c metabolite and FIG. 5B M4 metabolite).

### DETAILED DESCRIPTION OF THE INVENTION

Described herein are atropisomers of 2-(5-bromo-4-(4-cyclopropylnaphthalen-1-yl)-4H-1,2,4-triazol-3-ylthio)acetic acid, which are useful for decreasing uric acid levels.

Many organic compounds exist in optically active forms, i.e., they have the ability to rotate plane-polarized light. The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that they are mirror images of one another. Stereoisomers that are mirror images of one another may also be referred to as enantiomers, and a mixture of such isomers is often called an enantiomeric mixture. A 50:50 mixture of enantiomers is referred to as a racemic mixture or a racemate. Atropisomers are stereoisomers arising due to hindered rotation about a single bond, where energy differences create a barrier to rotation high enough to allow for isolation of individual conformers. Thus, Atropisomers exist in a thermally controlled equilibrium, differing from most other types of chiral structures, where interconversion requires chemical isomerization (i.e. breaking covalent bonds).

The energy barrier to thermal racemization of atropisomers may be determined by the steric hindrance to free rotation of one or more bonds forming a chiral axis. Certain biaryl compounds exhibit atropisomerism where rotation around an interannular bond lacking C2 symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biphenyl compounds may exhibit this type of conformational, rotational isomerism. Such biphenyls are enantiomeric, chiral atropisomers where the sp2-sp2 carbon-carbon, interannular bond between the phenyl rings has a sufficiently high energy barrier to prevent free rotation, and where substituents A≠B and A′≠B′ render the molecule asymmetric.

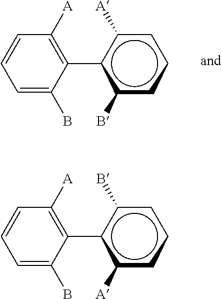

The steric interaction between A:A′, B:B′, and/or A:B′, B:A′ is large enough to make the planar conformation an energy maximum. Two non-planar, axially chiral enantiomers then exist as atropisomers when their interconversion is slow enough such that they can be isolated free of each other. By one definition, atropisomerism is defined to exist where the isomers have a half-life, t_{1/2}, of at least 1,000 seconds, which is a free energy barrier of 22.3 kcal mol⁻¹ (93.3kJ mol⁻¹) at 300K (Oki, M. “Recent Advances in Atropisomerism,” Topics in Stereochemistry (1983) 14:1). Bold lines and dashed lines in the figures shown above indicate those moieties, or portions of the molecule, which are sterically restricted due to a rotational energy barrier. Bolded moieties exist orthogonally above the plane of the page, and dashed moieties exist orthogonally below the plane of the page. The “flat” part of the molecule (the left ring in each of the two depicted biphenyls) is in the plane of the page. Compounds with axial chirality, such as chiral biphenyl rings, can be described using configurational nomenclature. Atropisomers often, though not always, have substituents ortho to the aryl-aryl bond that cause significant steric repulsion thereby hindering the rotation. Factors influencing the stability of individual atropisomers include: repulsive interactions (e.g. steric bulk) of substituents near the axis of rotation; the length and rigidity of the aryl-aryl bond; and whether there are pathways, other than thermal, to induce rotation.

### Stereochemical Assignment

Determining the axial stereochemistry of biaryl atropisomers can be accomplished by analysis of a Newman projection along the axis of hindered rotation.

The ortho substituents are assigned priority according to Cahn-Ingold-Prelog priority rules. Starting with the substituent of highest priority in the closest ring and moving along the shortest 90° path to the substituent of highest priority in the other ring (A to A′ in scheme 1 below), the absolute configuration is assigned P (plus) for clockwise and M (minus) for counterclockwise. In the example below, A has priority over A′ and B has priority over B′.

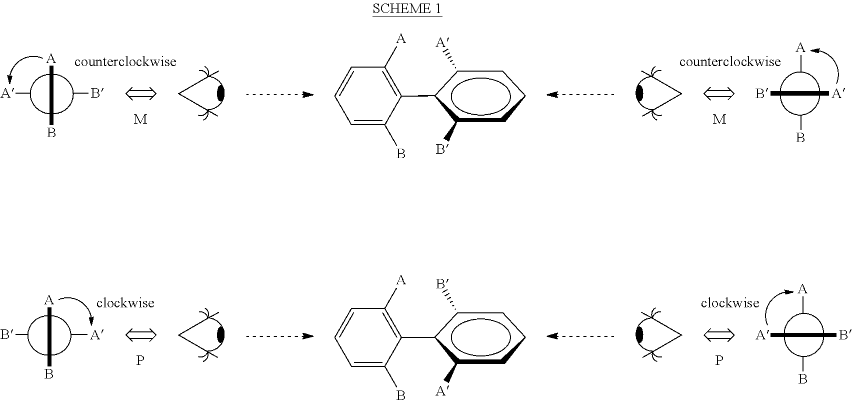

For a review of atropisomers, including their nomenclature, see “Directed Synthesis of Chiral Biaryl Compounds” by Bringmann et. al. in Angew. Chem. Int. Ed. 2005, 44, 5384-5427. Alternate methods for assigning absolute axial configuration have been contemplated; see for example U.S. Pat. No. 8,440,677, columns 8 and 9.

Stereoisomers of 2-(5-Bromo-4-(4-cyclopropylnaphthalen-1-yl)-4H-1,2,4-triazol-3-ylthio)acetic acid

The present invention relates to atropisomers of Compound (I) (2-(5-bromo-4-(4-cyclopropyl naphthalen-1-yl)-4H-1,2,4-triazol-3-ylthio)acetic acid), which are useful in decreasing uric acid levels.

2-(5-Bromo-4-(4-cyclopropylnaphthalen-1-yl)-4H-1,2,4-triazol-3-ylthio)acetic acid (Compound (I)) and related compounds are described in US Patent Application Publications 2008-0176850, US 2009-0197825, US 2010-0056464, US 2010-0056465, US 2010-0069645, and US 2010-0081827.

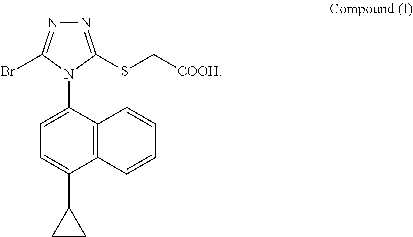 [SMILES: O=C(O)CSc1nnc(Br)n1-c1ccc(C2CC2)c2ccccc12]

### Stereochemistry of Compound (I)

In some embodiments the atropisomer of Compound (I) is (M)-2-(5-bromo-4-(4-cyclopropyl naphthalen-1-yl)-4H-1,2,4-triazol-3-ylthio)acetic acid as shown below:

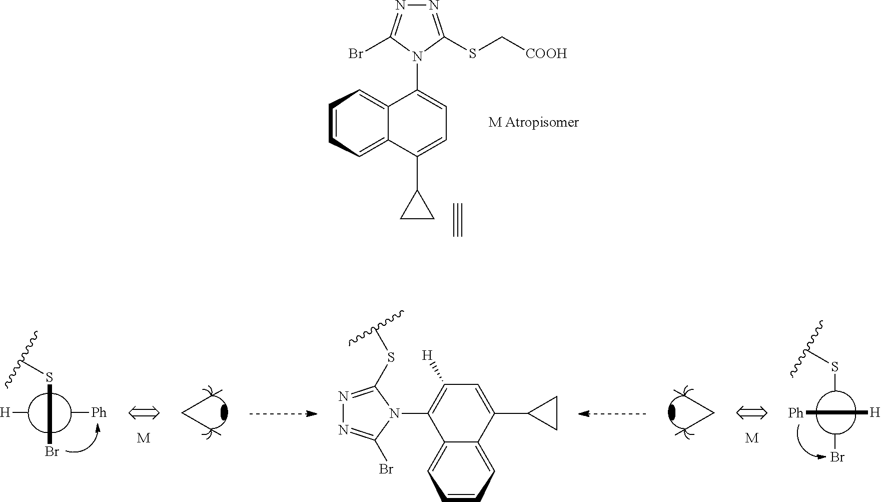

In some embodiments the atropisomer of Compound (I) is (P)-2-(5-bromo-4-(4-cyclopropyl naphthalen-1-yl)-4H-1,2,4-triazol-3-ylthio)acetic acid as shown below:

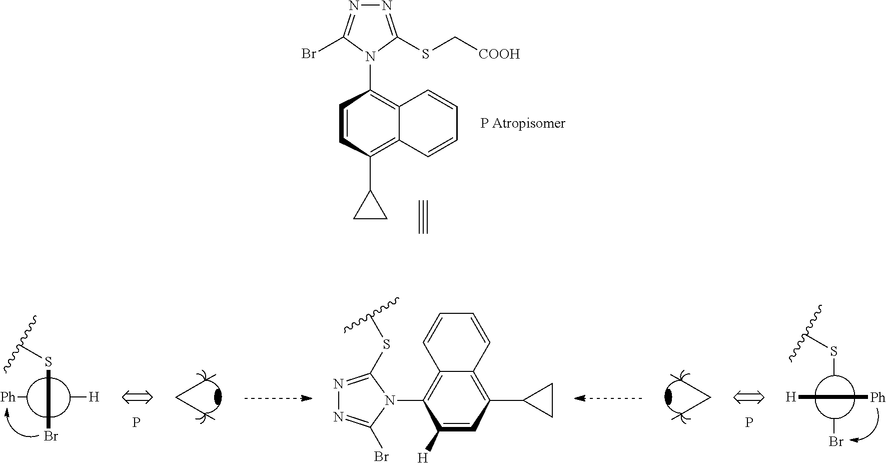

In some embodiments the atropisomer of Compound (I) is (−)-2-(5-bromo-4-(4-cyclopropyl naphthalen-1-yl)-4H-1,2,4-triazol-3-ylthio)acetic acid (atroisomer 1). In some embodiments the atropisomer of Compound (I) is (+)-2-(5-bromo-4-(4-cyclopropyl naphthalen-l-yl)-4H-1,2,4-triazol-3-ylthio)acetic acid (atropisomer 2).

Atropisomer 1

Atropisomer 1 or (−)-2-(5-bromo-4-(4-cyclopropyl naphthalen-1-yl)-4H-1,2,4-triazol-3-ylthio)acetic acid refers to the atropisomer of 2-(5-bromo-4-(4-cyclopropyl naphthalen-1-yl)-4H-1,2,4-triazol-3-ylthio)acetic acid that exhibits an optical rotation of about −9.1 deg. measured at 25° C., at a wavelength of 589 nm (D band is the result of the absorption by sodium atoms of light), in methanol (See table 2). In some embodiments, the optical rotation is between about −9.5 deg. and about −8.5 deg. In some embodiments, the optical rotation is between about −9.4 deg. and about −8.8 deg.

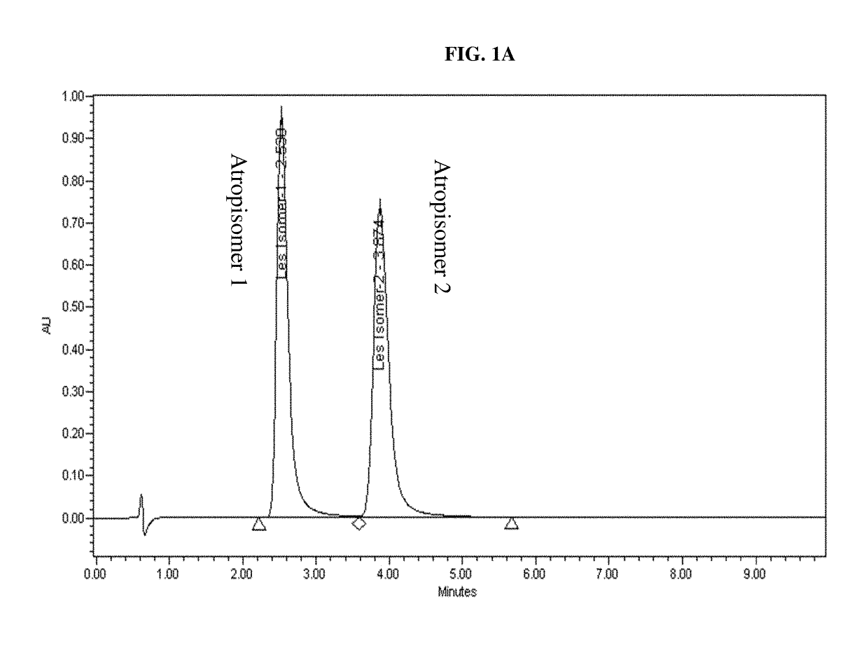

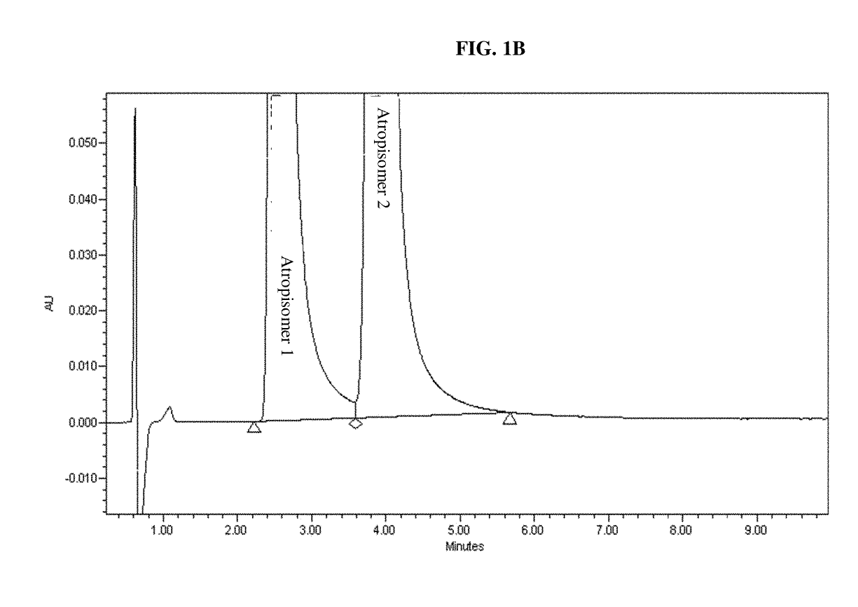

Atropisomer 1 or (−)-2-(5-bromo-4-(4-cyclopropyl naphthalen-1-yl)-4H-1,2,4-triazol-3-ylthio)acetic acid refers to the atropisomer of 2-(5-bromo-4-(4-cyclopropyl naphthalen-1-yl)-4H-1,2,4-triazol-3-ylthio)acetic acid that exhibits a retention time of about 2.7 minutes (atropisomer 1 as shown in FIG. 1A and 1B) as measured by the method of Example 2. In some embodiments, the retention time of atropisomer 1 is between about 2.2 and about 3.2 minutes as measured by the method of Example 2.

In some embodiments, the atropisomer (−)-2-(5-bromo-4-(4-cyclopropyl naphthalen-1-yl)-4H-1,2,4-triazol-3-ylthio)acetic acid is in enantiomeric excess. In some embodiments, the atropisomer (−)-2-(5-bromo-4-(4-cyclopropyl naphthalen-l-yl)-4H-1,2,4-triazol-3-ylthio)acetic acid is provided in at least 20%, 30%, 40%, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In other embodiments, the atropisomer (−)-2-(5-bromo-4-(4-cyclopropyl naphthalen-1-yl)-4H-1,2,4-triazol-3-ylthio)acetic acid is provided in greater than 20%, 30%, 40%, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In some embodiments, the atropisomer (−)-2-(5-bromo-4-(4-cyclopropyl naphthalen-1-yl)-4H-1,2,4-triazol-3-ylthio)acetic acid is in greater than 95% enantiomeric excess. In some embodiments, the atropisomer (−)-2-(5-bromo-4-(4-cyclopropyl naphthalen-1-yl)-4H-1,2,4-triazol-3-ylthio)acetic acid is in greater than 98% enantiomeric excess. In some embodiments, the atropisomer (−)-2-(5-bromo-4-(4-cyclopropyl naphthalen-l-yl)-4H-1,2,4-triazol-3-ylthio)acetic acid is in greater than 99% enantiomeric excess.

### Atropisomer 2

Atropisomer 1 or (+)-2-(5-bromo-4-(4-cyclopropyl naphthalen-1-yl)-4H-1,2,4-triazol-3-ylthio)acetic acid refers to the atropisomer of 2-(5-bromo-4-(4-cyclopropyl naphthalen-1-yl)-4H-1,2,4-triazol-3-ylthio)acetic acid that exhibit an optical rotation of about +7.8 deg. measured at 25 ° C., at a wavelength of 589 nm (D band is the result of the absorption by sodium atoms of light), in methanol (See table 2). In some embodiments, the optical rotation of between about +7.4 deg. and about +8.2 deg. In some embodiments, the optical rotation of between about +7.6 deg. and about +8.0 deg.

Atropisomer or (+)-2-(5-bromo-4-(4-cyclopropyl naphthalen-1-yl)-4H-1,2,4-triazol-3-ylthio)acetic acid refers to the atropisomer of 2-(5-bromo-4-(4-cyclopropyl naphthalen-1-yl)-4H-1,2,4-triazol-3-ylthio)acetic acid that exhibits a retention time of about 4.2 minutes (atropoisomer 2 as shown in FIG. 1A and 1B) as measured by the method of Example 2. In some embodiments, the retention time of atropisomer 2 is between about 3.7 and about 4.7 minutes as measured by the method of Example 2.

In some embodiments, the atropisomer (+)-2-(5-bromo-4-(4-cyclopropyl naphthalen-1-yl)-4H-1,2,4-triazol-3-ylthio)acetic acid is in enantiomeric excess. In some embodiments, the atropisomer (+)-2-(5-bromo-4-(4-cyclopropyl naphthalen-l-yl)-4H-1,2,4-triazol-3-ylthio)acetic acid is provided in at least 20%, 30%, 40%, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In other embodiments, the atropisomer (+)-2-(5-bromo-4-(4-cyclopropyl naphthalen-1-yl)-4H-1,2,4-triazol-3-ylthio)acetic acid is provided in greater than 20%, 30%, 40%, 50%, 55%, 60%, 65%, 70%, 75%, 80%, 85%, 88%, 90%, 91%, 92%, 93%, 94%, 95%, 96%, 97%, 98%, 99%, 99.5%, or 99.9% enantiomeric excess. In some embodiments, the atropisomer (+)-2-(5-bromo-4-(4-cyclopropyl naphthalen-1-yl)-4H-1,2,4-triazol-3-ylthio)acetic acid is in greater than 95% enantiomeric excess. In some embodiments, the atropisomer (+)-2-(5-bromo-4-(4-cyclopropyl naphthalen-1-yl)-4H-1,2,4-triazol-3-ylthio)acetic acid is in greater than 98% enantiomeric excess. In some embodiments, the atropisomer (+)-2-(5-bromo-4-(4-cyclopropyl naphthalen-l-yl)-4H-1,2,4-triazol-3-ylthio)acetic acid is in greater than 99% enantiomeric excess.

### Definitions

The terms “racemic mixture” and “racemate” refer to an equimolar mixture of two enantiomeric species, which is devoid of optical activity. In some embodiments, 2-(5-bromo-4-(4-cyclopropylnaphthalen-1-yl)-4H-1,2,4-triazol-3-ylthio)acetic acid is present as a racemic mixture.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

The term “enantiomers” as used herein, refers to two stereoisomers of a compound.

The term “atropisomers” refers to conformational stereoisomers which occur when rotation about a single bond in the molecule is prevented, or greatly slowed, as a result of steric interactions with other parts of the molecule and the substituents at both ends of the single bond are asymmetrical, i.e., they do not require a stereocenter. Where the rotational barrier about the single bond is high enough, and interconversion between conformations is slow enough, separation and isolation of the isomeric species may be permitted. Atropisomers are enantiomers without a single asymmetric atom. In some embodiments, the atropisomer is (−)-2-(5-bromo-4-(4-cyclopropylnaphthalen-1-yl)-4H-1,2,4-triazol-3-ylthio)acetic acid. In some embodiments, the atropisomer is (+)-2-(5-bromo-4-(4-cyclopropylnaphthalen-1-yl)-4H-1,2,4-triazol-3-ylthio)acetic acid. In some embodiments, the atropisomer is (M)-2-(5-bromo-4-(4-cyclopropylnaphthalen-1-yl)-4H-1,2,4-triazol-3-ylthio)acetic acid. In some embodiments, the atropisomer is (P)-2-(5-bromo-4-(4-cyclopropylnaphthalen-1-yl)-4H-1,2,4-triazol-3-ylthio)acetic acid.

The term “enantiomeric excess,” refers to the percent excess of one enantiomer compared to that of the other enantiomer in a mixture. It can be calculated using the following equation: enantiomeric excess=((R−S)/(R+S))×100=%(R*)−%(S*), wherein R and S are the number of moles of each enantiomer in the mixture, and R* and S* are the respective mole fractions of the enantiomers in the mixture. For example, for a mixture with 87% R enantiomer and 13% S enantiomer, the enantiomeric excess is 74%.

The term “subject”, as used herein in reference to individuals suffering from a disorder, and the like, encompasses mammals and non-mammals. In one embodiment of the methods and compositions provided herein, the mammal is a human.

The terms “effective amount”, “therapeutically effective amount” or “pharmaceutically effective amount” as used herein, refer to an amount of at least one agent or compound being administered that is sufficient to treat or prevent the particular disease or condition. The result is the reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case is determined using techniques such as a dose escalation study.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

The term “about” refers to ±10% of a stated number or value.

### Modulating URAT-1 Activity

Also described herein are methods of modulating URAT-1 activity by contacting URAT-1 with an amount of an atropisomer of 2-(5-bromo-4-(4-cyclopropylnaphthalen-1-yl)-4H-1,2,4-triazol-3-ylthio)acetic acid, as described herein, sufficient to modulate the activity of URAT-1. The term “modulate” refers to either inhibiting or activating URAT-1 activity. In some embodiments are provided methods of inhibiting URAT-1 activity by contacting URAT-1 with an amount of an atropisomer of 2-(5-bromo-4-(4-cyclopropylnaphthalen-1-yl)-4H-1,2,4-triazol-3-ylthio)acetic acid, as described herein, sufficient to inhibit the activity of URAT-1. In some embodiments are provided methods of inhibiting URAT-1 activity in a solution by contacting said solution with an amount of an atropisomer of 2-(5-bromo-4-(4-cyclopropylnaphthalen-1-yl)-4H-1,2,4-triazol-3-ylthio)acetic acid, as described herein sufficient to inhibit the activity of URAT-1 in said solution. In some embodiments are provided methods of inhibiting URAT-1 activity in a cell by contacting said cell with an amount of an atropisomer of 2-(5-bromo-4-(4-cyclopropylnaphthalen-1-yl)-4H-1,2,4-triazol-3-ylthio)acetic acid, as described herein, sufficient to inhibit the activity of URAT-1 in said cell. In some embodiments are provided methods of inhibiting URAT-1 activity in a tissue by contacting said tissue with an amount of an atropisomer of 2-(5-bromo-4-(4-cyclopropylnaphthalen-1-yl)-4H-1,2,4-triazol-3-ylthio)acetic acid, as described herein, sufficient to inhibit the activity of URAT-1 in said tissue. In some embodiments are provided methods of inhibiting URAT-1 activity in blood by contacting the blood with an amount of an atropisomer of 2-(5-bromo-4-(4-cyclopropylnaphthalen-1-yl)-4H-1,2,4-triazol-3-ylthio)acetic acid, as described herein, sufficient to inhibit the activity of URAT-1 in blood. In some embodiments are provided methods of inhibiting URAT-1 activity in plasma by contacting the plasma with an amount of an atropisomer of 2-(5-bromo-4-(4-cyclopropylnaphthalen-1-yl)-4H-1,2,4-triazol-3-ylthio)acetic acid, as described herein, sufficient to inhibit the activity of URAT-1 in plasma. In some embodiments are provided methods of inhibiting URAT-1 activity in an animal by contacting said animal with an amount of an atropisomer of 2-(5-bromo-4-(4-cyclopropylnaphthalen-1-yl)-4H-1,2,4-triazol-3-ylthio)acetic acid, as described herein sufficient to inhibit the activity of URAT-1 in said animal. In some embodiments are provided methods of inhibiting URAT-1 activity in a mammal by contacting said mammal with an amount of an atropisomer of 2-(5-bromo-4-(4-cyclopropylnaphthalen-1-yl)-4H-1,2,4-triazol-3-ylthio)acetic acid, as described herein sufficient to inhibit the activity of URAT-1 in said mammal. In some embodiments are provided methods of inhibiting URAT-1 activity in a human by contacting said human with an amount of an atropisomer of 2-(5-bromo-4-(4-cyclopropylnaphthalen-1-yl)-4H-1,2,4-triazol-3-ylthio)acetic acid, as described herein, sufficient to inhibit the activity of URAT-1 in said human.

### Pharmaceutical Compositions

Described herein are pharmaceutical compositions comprising an effective amount of an atropisomer of 2-(5-bromo-4-(4-cyclopropylnaphthalen-1-yl)-4H-1,2,4-triazol-3-ylthio)acetic acid, as described herein. In some embodiments, the pharmaceutical compositions comprise an effective amount of an atropisomer of 2-(5-bromo-4-(4-cyclopropylnaphthalen-1-yl)-4H-1,2,4-triazol-3-ylthio)acetic acid, as described herein, and at least one pharmaceutically acceptable carrier. In some embodiments, the pharmaceutical compositions comprise an effective amount of (−)-2-(5-bromo-4-(4-cyclopropylnaphthalen-1-yl)-4H-1,2,4-triazol-3-ylthio)acetic acid, as described herein, and at least one pharmaceutically acceptable carrier. In some embodiments, the pharmaceutical compositions comprise an effective amount of (+)-2-(5-bromo-4-(4-cyclopropylnaphthalen-1-yl)-4H-1,2,4-triazol-3-ylthio)acetic acid, as described herein, and at least one pharmaceutically acceptable carrier. In some embodiments, the pharmaceutical compositions comprise an effective amount of a combination of (−)-2-(5-bromo-4-(4-cyclopropylnaphthalen-1-yl)-4H-1,2,4-triazol-3-ylthio)acetic acid and (+)-2-(5-bromo-4-(4-cyclopropylnaphthalen-1-yl)-4H-1,2,4-triazol-3-ylthio)acetic acid, as described herein, and at least one pharmaceutically acceptable carrier. In some embodiments the pharmaceutical compositions are for the treatment of disorders. In some embodiments the pharmaceutical compositions are for the treatment of disorders in a mammal. In some embodiments the pharmaceutical compositions are for the treatment of disorders in a human. In some embodiments the pharmaceutical compositions are for the treatment or prophylaxis of disorders of uric acid metabolism. In some embodiments the pharmaceutical compositions are for the treatment or prophylaxis of hyperuricemia. In some embodiments the pharmaceutical compositions are for the treatment or prophylaxis of gout.

### Modes of Administration, Formulations and Dosage Forms

Described herein are pharmaceutical compositions comprising an atropisomer of 2(5-bromo-4-(4-cyclopropylnaphthalen-1-yl)-4H-1,2,4-triazol-3-ylthio)acetic acid, as described herein. The compound, compound forms and compositions described herein are administered either alone, or in combination with, pharmaceutically acceptable carriers, excipients, or diluents in a pharmaceutical composition, according to standard pharmaceutical practice. Administration is effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route depends upon, for example, the condition and disorder of the recipient. Those of skill in the art will be familiar with administration techniques that can be employed with the compounds, compound forms, compositions and methods described herein. By way of example only, the compounds, compound forms and compositions described herein are, in some embodiments, administered locally to the area in need of treatment, by for example, local infusion during surgery, topical application such as creams or ointments, injection, catheter, or implant, said implant made for example, out of a porous, non-porous, or gelatinous material, including membranes, such as sialastic membranes, or fibers. The administration is, in some embodiments, by direct injection at the site of a diseased tissue or organ.

The pharmaceutical compositions described herein are, for example, in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition is, in some embodiments, in unit dosage forms suitable for single administration of precise dosages. Pharmaceutical compositions include a compound or compound form as described herein as an active ingredient, and a conventional pharmaceutical carrier or excipient. In some embodiments these compositions include other or additional medicinal or pharmaceutical agents, carriers, adjuvants, etc.

Pharmaceutical compositions are conveniently presented in unit dosage form. In some embodiments, they are prepared with a specific amount of active compound by any of the methods well known or apparent to those skilled in the pharmaceutical arts.

### Doses

The amount of pharmaceutical compositions administered will firstly be dependent on the mammal being treated. In the instances where pharmaceutical compositions are administered to a human subject, the daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, drug combination, and the discretion of the prescribing physician. Also, the route of administration vary depending on the condition and its severity. The pharmaceutical composition is, in some embodiments, in unit dosage form. In such form, the preparation is subdivided into unit doses containing appropriate quantities of the active component, e.g., an effective amount to achieve the desired purpose. Determination of the proper dosage for a particular situation is within the skill of the art. For convenience, in some embodiments, the total daily dosage is divided and administered in portions during the day if desired. The amount and frequency of administration will be regulated according to the judgment of the attending clinician physician considering such factors as described above. Thus the amount of pharmaceutical composition to be administered is variable depending upon the circumstances. The quantity of active compound in a unit dose of preparation is, in some embodiments, varied or adjusted from about 250 to about 400 mg, or from about 25 mg to about 200 mg, according to the particular application. In some instances the particular therapeutic dosage is about 200 mg, about 150 mg, about 100 mg, about 50 mg, about 25 mg, or about 20 mg. In some instances, the dosage of the atropisomer is less than the dosage of the corresponding racemic mixture. In some instances, the dosage of the atropisomer is about ½, about ⅓, about ¼, about ⅕, about ⅙, about 1/7, about ⅛, about 1/9, or about 1/10 of the dosage of the corresponding racemic mixture. In some instances, dosage levels below the lower limit of the aforesaid range are more than adequate, while in other cases still larger doses are employed without causing any harmful side effect, e.g. by dividing such larger doses into several small doses for administration throughout the day. In combinational applications in which the compound is not the sole therapy, it is possible to administer lesser amounts of compound and still have therapeutic or prophylactic effect.

### Combination Therapies

The compounds and compound forms described herein are administered as a sole therapy or in combination with another therapy or therapies.

By way of example only, if one of the side effects experienced by a patient upon receiving a compound or compound form as described herein is hypertension, then it may be appropriate to administer an anti-hypertensive agent in combination with the compound. Or, by way of example only, the therapeutic effectiveness of a compound or compound form as described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering a compound or compound form as described herein with another therapeutic agent (which also includes a therapeutic regimen) that also has therapeutic benefit. Regardless of the disease, disorder or condition being treated, the overall benefit experienced by the patient may simply be additive of the two therapeutic agents or the patient may experience a synergistic benefit.

In the instances where the compounds or compound forms as described herein are administered with other therapeutic agents, they need not be administered in the same pharmaceutical composition as other therapeutic agents, and may, because of different physical and chemical characteristics, be administered by a different route. For example, the compound or compound form as described herein may be administered orally to generate and maintain good blood levels thereof, while the other therapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The compounds, compound forms and compositions described herein (and where appropriate other chemotherapeutic agent) may be administered concurrently (e.g., simultaneously, essentially simultaneously or within the same treatment protocol) sequentially or separately, depending upon the nature of the disease, the condition of the patient, and the actual choice of other chemotherapeutic agent to be administered. For combinational applications and uses, the compounds, compound forms and compositions described herein and the chemotherapeutic agent need not be administered simultaneously or essentially simultaneously. Thus, the compounds, compound forms and compositions as described herein may be administered first followed by the administration of the chemotherapeutic agent; or the chemotherapeutic agent may be administered first followed by the administration of the compounds, compound forms and compositions as described herein. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent may be administered first, especially if it is a cytotoxic agent, and then the treatment continued with the administration of the compounds, compound forms and compositions as described herein followed, where determined advantageous, by the administration of the chemotherapeutic agent, and so on until the treatment protocol is complete. Thus, in accordance with experience and knowledge, the practicing physician can modify each administration protocol for treatment according to the individual patient's needs, as the treatment proceeds. The attending clinician, in judging whether treatment is effective at the dosage administered, will consider the general well-being of the patient as well as more definite signs such as relief of disease-related symptoms. Relief of disease-related symptoms such as pain, and improvement in overall condition can also be used to help judge effectiveness of treatment.

Specific, non-limiting examples of possible combination therapies include use of the compounds, compound forms and compositions described herein with Febuxostat, Allopurinol, Probenecid, Sulfinpyrazone, Losartan, Fenofibrate, Benzbromarone or PNP-inhibitors (such as, but not limited to Forodesine, BCX-1777 or BCX-4208). This list should not be construed to be closed, but should instead serve as an illustrative example common to the relevant therapeutic area at present. Moreover, combination regimens may include a variety of routes of administration, including but not limited to oral, intravenous, intraocular, subcutaneous, dermal, and inhaled topical.

### Diseases

Described herein are methods of treating a disease or disorder in an individual suffering from the disease or disorder comprising administering to said individual an effective amount of an atropisomer as described herein of 2-(5-bromo-4-(4-cyclopropylnaphthalen-1-yl)-4H-1,2,4-triazol-3-ylthio)acetic acid.

Also described herein are methods of preventing a disease or disorder in an individual comprising administering to said individual an effective amount of an atropisomer as described herein of 2-(5-bromo-4-(4-cyclopropylnaphthalen-1-yl)-4H-1,2,4-triazol-3-ylthio)acetic acid.

The invention extends to the use of the compounds, compound forms and compositions described herein, in the manufacture of a medicament for treating or preventing a disease or disorder.

In some embodiments, the disease or disorder is hyperuricemia. In certain instances, hyperuricemia is characterized by higher than normal blood levels of uric acid, sustained over long periods of time. In certain instances, increased blood urate levels may be due to enhanced uric acid production (˜10-20%) and/or reduced renal excretion (˜80-90%) of uric acid. In certain instances, causes of hyperuricemia may include obesity/weight gain, excessive alcohol use, excessive dietary purine intake (foods such as shellfish, fish roe, scallops, peas lentils, beans and red meat, particularly offal-brains, kidneys, tripe, liver), certain medications, including low-dose aspirin, diuretics, niacin, cyclosporine, pyrazinamide, ethambutol, some high blood pressure drugs and some cancer chemotherapeutics, immunosuppressive and cytotoxic agents, specific disease states, particularly those associated with a high cell turnover rate (such as malignancy, leukemia, lymphoma or psoriasis), and also including high blood pressure, hemoglobin diseases, hemolytic anemia, sickle cell anemia, various nephropathies, myeloproliferative and lymphoproliferative diseases, hyperparathyroidism, renal disease, conditions associated with insulin resistance and diabetes mellitus, and in transplant recipients, and possibly heart disease, inherited enzyme defects, abnormal kidney function (e.g. increased ATP turn over, reduced glomerular urate filtration) and exposure to lead (plumbism or “saturnine gout”).

In certain instances, hyperuricemia may be asymptomatic, though is associated with the following conditions: gout, gouty arthritis, uric acid stones in the urinary tract (urolithiasis), deposits of uric acid in the soft tissue (tophi), deposits of uric acid in the kidneys (uric acid nephropathy), and impaired kidney function, possibly leading to chronic and acute renal failure.

In further or additional embodiments, the disease or disorder is gout, which is a condition that results from uric acid crystals depositing in tissues of the body. It is often related to an inherited abnormality in the body's ability to process uric acid, but may also be exacerbated by a purine rich diet. Defective uric acid processing may lead to elevated levels of uric acid in the blood causing recurring attacks of joint inflammation (arthritis), uric acid deposits in and around the joints, tophaceous gout, the formation of tophi, decreased kidney function, and kidney stones. Approximately 3-5 million people in the United States suffer from attacks of gout with attacks more prevalent in men than in women. In certain instances, gout is one of the most common forms of arthritis, accounting for approximately 5% of all arthritis cases. In certain instances, kidney failure and urolithiasis occur in 10-18% of individuals with gout and are common sources of morbidity and mortality from the disease.

Gout is associated with hyperuricemia. In certain instances, individuals suffering from gout excrete approximately 40% less uric acid than non-gouty individuals for any given plasma urate concentration. In certain instances, urate levels increase until the saturation point is reached. In certain instances, precipitation of urate crystals occurs when the saturation point is reached. In certain instances, these hardened, crystallized deposits (tophi) form in the joints and skin, causing joint inflammation (arthritis). In certain instances, deposits are be made in the joint fluid (synovial fluid) and/or joint lining (synovial lining). Common areas for these deposits are the large toe, feet, ankles and hands (less common areas include the ears and eyes). In certain instances, the skin around an affected joint becomes red and shiny with the affected area being tender and painful to touch. In certain instances, gout attacks increase in frequency. In certain instances, untreated acute gout attacks lead to permanent joint damage and disability. In certain instances, tissue deposition of urate leads to: acute inflammatory arthritis, chronic arthritis, deposition of urate crystals in renal parenchyma and urolithiasis. In certain instances, the incidence of gouty arthritis increases 5 fold in individuals with serum urate levels of 7 to 8.9 mg/dL and up to 50 fold in individuals with levels >9mg/dL (530 μmol/L). In certain instances, individuals with gout develop renal insufficiency and end stage renal disease (i.e., “gouty nephropathy”). In certain instances, gouty nephropathy is characterized by a chronic interstitial nephropathy, which is promoted by medullary deposition of monosodium urate.

In certain instances, gout includes painful attacks of acute, monarticular, inflammatory arthritis, deposition of urate crystals in joints, deposition of urate crystals in renal parenchyma, urolithiasis (formation of calculus in the urinary tract), and nephrolithiasis (formation of kidney stones). In certain instances, secondary gout occurs in individuals with cancer, particularly leukemia, and those with other blood diseases (e.g. polycythemia, myeloid metaplasia, etc).

In certain instances, attacks of gout develop very quickly, frequently the first attack occurring at night. In certain instances, symptoms include sudden, severe joint pain and extreme tenderness in the joint area, joint swelling and shiny red or purple skin around the joint. In certain instances, the attacks are infrequent lasting 5-10 days, with no symptoms between episodes. In certain instances, attacks become more frequent and last longer, especially if the disease is not controlled. In certain instances, episodes damage the affected joint(s) resulting in stiffness, swelling, limited motion and/or persistent mild to moderate pain.

Plumbism or “saturnine gout,” is a lead-induced hyperuricemia that results from lead inhibition of tubular urate transport causing decreased renal excretion of uric acid. In certain instances, more than 50% of individuals suffering from lead nephropathy suffer from gout. In certain instances, acute attacks of saturnine gout occur in the knee more frequently than the big toe. In certain instances, renal disease is more frequent and more severe in saturnine gout than in primary gout. In certain instances, treatment consists of excluding the individual from further exposure to lead, the use of chelating agents to remove lead, and control of acute gouty arthritis and hyperuricemia. In certain instances, saturnine gout is characterized by less frequent attacks than primary gout. In certain instances, lead-associated gout occurs in pre-menopausal women, an uncommon occurrence in non lead-associated gout.

In certain instances, Lesch-Nyhan syndrome (LNS or Nyhan's syndrome) affects about one in 100,000 live births. In certain instances, LNS is caused by a genetic deficiency of the enzyme hypoxanthine-guanine phosphoribosyltransferase (HGPRT). In certain instances, LNS is an X-linked recessive disease. In certain instances, LNS is present at birth in baby boys. In certain instances, the disease leads to severe gout, poor muscle control, and moderate mental retardation, which appear in the first year of life. In certain instances, the disease also results in self-mutilating behaviors (e.g., lip and finger biting, head banging) beginning in the second year of life. In certain instances, the disease also results in gout-like swelling in the joints and severe kidney problems. In certain instances, the disease leads neurological symptoms include facial grimacing, involuntary writhing, and repetitive movements of the arms and legs similar to those seen in Huntington's disease. The prognosis for individuals with LNS is poor. In certain instances, the life expectancy of an untreated individual with LNS is less than about 5 years. In certain instances, the life expectancy of a treated individual with LNS is greater than about 40 years of age.

In certain instances, hyperuricemia is found in individuals with cardiovascular disease (CVD) and/or renal disease. In certain instances, hyperuricemia is found in individuals with prehypertension, hypertension, increased proximal sodium reabsorption, microalbuminuria, proteinuria, kidney disease, obesity, hypertriglyceridemia, low high-density lipoprotein cholesterol, hyperinsulinemia, hyperleptinemia, hypoadiponectinemia, peripheral, carotid and coronary artery disease, atherosclerosis, congestive heart failure, stroke, tumor lysis syndrome, endothelial dysfunction, oxidative stress, elevated renin levels, elevated endothelin levels, and/or elevated C-reactive protein levels. In certain instances, hyperuricemia is found in individuals with obesity (e.g., central obesity), high blood pressure, hyperlipidemia, and/or impaired fasting glucose. In certain instances, hyperuricemia is found in individuals with metabolic syndrome. In certain instances, gouty arthritis is indicative of an increased risk of acute myocardial infarction. In some embodiments, administration of a compound described herein to an individual are useful for decreasing the likelihood of a clinical event associated with a disease or condition linked to hyperuricemia, including, but not limited to, prehypertension, hypertension, increased proximal sodium reabsorption, microalbuminuria, proteinuria, kidney disease, obesity, hypertriglyceridemia, low high-density lipoprotein cholesterol, hyperinsulinemia, hyperleptinemia, hypoadiponectinemia, peripheral, carotid and coronary artery disease, atherosclerosis, congestive heart failure, stroke, tumor lysis syndrome, endothelial dysfunction, oxidative stress, elevated renin levels, elevated endothelin levels, and/or elevated C-reactive protein levels.

In some embodiments, a compound or compound form as described herein is administered to an individual suffering from a disease or condition requiring treatment with a diuretic. In some embodiments, a compound or compound form as described herein is administered to an individual suffering from a disease or condition requiring treatment with a diuretic, wherein the diuretic causes renal retention of urate. In some embodiments, the disease or condition is congestive heart failure or essential hypertension.

In some embodiments, administration of a compound or compound form as described herein to an individual is useful for improving motility or improving quality of life.

In some embodiments, administration of a compound or compound form as described herein to an individual is useful for treating or decreasing the side effects of cancer treatment.

In some embodiments, administration of a compound or compound form as described herein to an individual is useful for decreasing kidney toxicity of cis-platin.

In certain instances, gout is treated by lowering the production of uric acid. In certain instances, gout is treated by increasing the excretion of uric acid. In certain instances, gout is treated by a URAT 1 inhibitor, a xanthine oxidase inhibitor, a xanthine dehydrogenase inhibitor, a xanthine oxidoreductase inhibitor, a purine nucleoside phosphorylase (PNP) inhibitor, a uric acid transporter (URAT) inhibitor, a glucose transporter (GLUT) inhibitor, a GLUT-9 inhibitor, a solute carrier family 2 (facilitated glucose transporter), member 9 (SLC2A9) inhibitor, an organic anion transporter (OAT) inhibitor, an OAT-4 inhibitor, or combinations thereof. In general, the goals of gout treatment are to i) reduce the pain, swelling and duration of an acute attack, and ii) prevent future attacks and joint damage. In certain instances, gout attacks are treated successfully using a combination of treatments. In certain instances, gout is one of the most treatable forms of arthritis.

i) Treating the gout attack. In certain instances, the pain and swelling associated with an acute attack of gout can be addressed with medications such as acetaminophen, steroids, nonsteroidal anti-inflammatory drugs (NSAIDs), adrenocorticotropic hormone (ACTH) or colchicine. In certain instances, proper medication controls gout within 12 to 24 hours and treatment is stopped after a few days. In certain instances, medication is used in conjunction with rest, increased fluid intake, ice-packs, elevation and/or protection of the affected area/s. In certain instances, the aforementioned treatments do not prevent recurrent attacks and they do not affect the underlying diseases of abnormal uric acid metabolism.

ii) Preventing future attacks. In certain instances, reducing serum uric acid levels below the saturation level is the goal for preventing further gout attacks. In some cases, this is achieved by decreasing uric acid production (e.g. allopurinol), or increasing uric acid excretion with uricosuric agents (e.g. probenecid, sulfinpyrazone, benzbromarone).

In certain instances, allopurinol inhibits uric acid formation, resulting in a reduction in both the serum and urinary uric acid levels and becomes fully effective after 2 to 3 months.

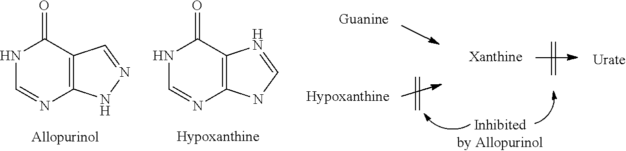

In certain instances, allopurinol is a structural analogue of hypoxanthine, (differing only in the transposition of the carbon and nitrogen atoms at positions 7 and 8), which inhibits the action of xanthine oxidase, the enzyme responsible for the conversion of hypoxanthine to xanthine, and xanthine to uric acid. In certain instances, it is metabolized to the corresponding xanthine analogue, alloxanthine (oxypurinol), which is also an inhibitor of xanthine oxidase. In certain instances, alloxanthine, though more potent in inhibiting xanthine oxidase, is less pharmaceutically acceptable due to low oral bioavailability. In certain instances, fatal reactions due to hypersensitivity, bone marrow suppression, hepatitis, and vasculitis have been reported with Allopurinol. In certain instances, the incidence of side effects may total 20% of all individuals treated with the drug. Treatment for diseases of uric acid metabolism has not evolved significantly in the following two decades since the introduction of allopurinol.

In certain instances, uricosuric agents (e.g., probenecid, sulfinpyrazone, and benzbromarone) increase uric acid excretion. In certain instances, probenecid causes an increase in uric acid secretion by the renal tubules and, when used chronically, mobilizes body stores of urate. In certain instances, 25-50% of individuals treated with probenecid fail to achieve reduction of serum uric acid levels <6 mg/dL. In certain instances, insensitivity to probenecid results from drug intolerance, concomitant salicylate ingestion, and renal impairment. In certain instances, one-third of the individuals develop intolerance to probenecid. In certain instances, administration of uricosuric agents also results in urinary calculus, gastrointestinal obstruction, jaundice and anemia.

Successful treatment aims to reduce both the pain associated with acute gout flare and long-term damage to the affected joints Therapeutic goals include providing rapid and safe pain relief, preventing further attacks, preventing the formation of tophi and subsequent arthritis, and avoiding exacerbating other medical conditions. Initiation of treatment depends upon the underlying causes of hyperuricemia, such as renal function, diet, and medications. While gout is a treatable condition, there are limited treatments available for managing acute and chronic gout and a number of adverse effects are associated with current therapies. Medication treatment of gout includes pain management, prevention or decrease in joint inflammation during an acute gouty attack, and chronic long-term therapy to maintain decreased serum uric acid levels.

Nonsteroidal anti-inflammatory drugs (NSAIDs) are effective anti-inflammatory medications for acute gout but are frequently associated with irritation of the gastrointestinal (GI) system, ulceration of the stomach and intestines, and occasionally intestinal bleeding. Colchicine for acute gout is most commonly administered orally as tablets (every 1-2 hours until there is significant improvement in pain or the patient develops GI side effects such as severe diarrhea, nausea and vomiting), or intravenously. Corticosteroids, given in short courses, can be administered orally or injected directly into the inflamed joint.

Medications are available for reducing blood uric acid levels that either increase renal excretion of uric acid by inhibiting re-uptake or reduce production of uric acid by blockade of xanthine oxidase. These medicines are generally not initiated until after the inflammation from acute gouty arthritis has subsided because they may intensify the attack. If they are already being taken prior to the attack, they are continued and only adjusted after the attack has resolved. Since many subjects with elevated blood uric acid levels may not develop gouty attacks or kidney stones, the decision for prolonged treatment with uric acid-lowering medications is individualized.

### Kits

The compounds, compound forms, compositions and methods described herein provide kits for the treatment of diseases and disorders, such as the ones described herein. These kits comprise a compound, compound form, compounds, compound forms or compositions described herein in a container and, optionally, instructions teaching the use of the kit according to the various methods and approaches described herein. Such kits, in some embodiments,also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein are provided, marketed and/or promoted to health providers, including physicians, nurses, pharmacists, formulary officials, and the like. Kits are also, in some embodiments, marketed directly to the consumer.

Provided in certain embodiments, are compositions or kits comprising an atropisomer of 2-(5-bromo-4-(4-cyclopropylnaphthalen-1-yl)-4H-1,2,4-triazol-3-ylthio)acetate, a double low density polyethylene plastic bag, and an HDPE container. In further embodiments, the composition or kit further comprises a foil bag (e.g., an anhydrous foil bag, such as a heat sealed anhydrous foil bag). In some embodiments, the composition or kit further comprises a desiccant; in still other embodiments, a desiccant is not necessary and/or present. In some instances, such packing improves the stability of the atropisomer of 2-(5-bromo-4-(4-cyclopropylnaphthalen-1-yl)-4H-1,2,4-triazol-3-ylthio)acetate.

In some embodiments, the compounds, compound forms and pharmaceutical compositions described herein are utilized for diagnostics and as research reagents. For example, in some embodiments, the compounds, compound forms and pharmaceutical compositions, either alone or in combination with other compounds, are used as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. These analyses are performed on stimulated or unstimulated cells and in the presence or absence of other compounds which affect expression patterns.

Besides being useful for human treatment, the compounds, compound forms and pharmaceutical compositions described herein are also useful for veterinary treatment of animals.

The examples and preparations provided below further illustrate and exemplify the compounds of the present invention and methods of preparing such compounds. It is to be understood that the scope of the present invention is not limited in any way by the scope of the following examples and preparations.

### EXAMPLES

### Example 1

### Computational Analysis

The restricted rotation around the naphthalene-triazole bond [symbol] of Compound (I) (2-(5-bromo-4-(4-cyclopropylnaphthalen-1-yl)-4H-1,2,4-triazol-3-ylthio)acetic acid) and Compound (II) (ethyl 2-(4-(4-cyclopropylnaphthalen-1-yl)-4H-1,2,4-triazol-3-ylthio)acetate) were studied using two computational methodologies. Compounds with rotational barriers greater than 20 kcal/mol have the potential to form atropisomers.

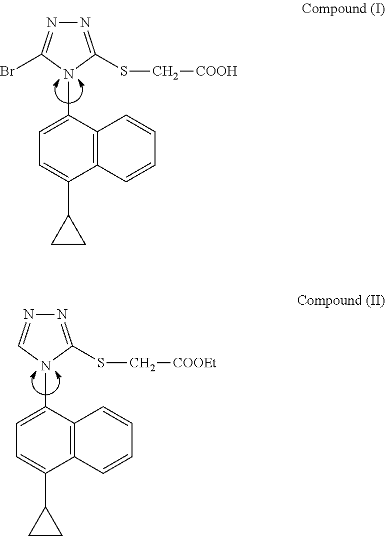 [SMILES: O=C(O)CSc1nnc(Br)n1-c1ccc(C2CC2)c2ccccc12 ; CCOC(=O)CSc1nncn1-c1ccc(C2CC2)c2ccccc12]

### Computational Analysis—Density Functional Theory (DFT)

Density functional theory (DFT) quantum chemical calculations employed a relaxed torsional scan, at 15° intervals, of the hindered naphthalene-triazole bond rotation in the gas phase, (see for example LaPlante et al, “Revealing atropisomer axial chirality in drug discovery”, Chem Med Chem. 2011, 6 (3), 505-513). Gaussian 09 (revision D02) was used and the level of theory adopted was B3LYP-D3/6-31+G(d,p) (except for Br, where the LANL2DZ effective core potential was used).

### Computational Analysis—Metadynamics

Metadynamics (an efficient molecular dynamics simulation protocol) Desmond software was implemented in the Schrodinger suite using the OPLS-2005 force-field. The results of these analyses are presented in the table 1 below:

TABLE 1

| | Computa- | Predicted | | |
|---|---|---|---|---|
| Com- | tional | barrier to | | |
| pound | method | interconversion | Half-life | |
| (I) | DFT | 30 kcal/mol | 860 | days (37° C.) |
| (I) | metadynamics | 27 kcal/mol | 6.5 | days (37° C.) |
| (II) | DFT | 14 kcal/mol | 1 | millisecond |
| (II) | metadynamics | 15 kcal/mol | 5 | milliseconds (25° C.) |

These computational experiments indicate that individual atropisomers of Compound (I) are likely to exist at room temperature and their interconversion at 37° C., or lower, is unlikely.

### Example 2

### Determination of Enantiomeric Purity and Enantiomeric Excess

Compound (I) (racemic mixture) was chromatographed on a chiral column, and the enantiomeric purity and excess of the separate atropisomers determined by the peak areas in the HPLC traces.

### Step 1: Preparation of a Working Standard

Compound (I) (approximately 50 mg) was accurately weighed into a 100 mL volumetric flask, using a small disposable anti-static polypropylene weighing funnel (TradeWinds Direct). Acetonitrile (HPLC grade) was added to about 80% volume and the compound dissolved (sonicate and swirl, if necessary). The solution was diluted to volume with acetonitrile and mixed well to yield a standard with a target concentration of approximately 0.5 mg/mL (0.25 mg/mL for each atropisomer, assuming an approximately 50:50 mixture).

### Step 2: Preparation of a Practical Limit of Quantitation (PLOQ) Standard

The working standard solution (see STEP 2 above) was diluted by transferring 5 mL to a 100 mL volumetric flask and diluted to volume with acetonitrile with thorough mixing, to provide a concentration of the PLOQ stock solution of 25 μg/mL of Compound (I) (12.5 mg/mL for each atropisomer).

The 25 μg/mL solution was further diluted by transferring 2.0 mL to a 100 mL volumetric flask and diluting to volume with acetonitrile with thorough mixing. Final concentration of the PLOQ solution was 0.5 μg/mL (0.25 μg/mL for each atropisomer).

### Step 3: Preparation of a Test Sample

Compound (I) atropisomer (approximately 25 mg) was accurately weighed into a 100 mL volumetric flask. Acetonitrile was added to about 80% volume and the compound dissolved (sonicate and swirl, if necessary). The solution was diluted to volume with acetonitrile and mixed well to yield samples with a target concentration of about 0.25 mg/mL.

### Step 4: HPLC

The samples were run through an HPLC system, as follows:
System: Agilent 1100 or 1200 Series HPLC System
Detector: 226 nm, Bw 8 nm/Reference 400 nm, Bw 50 nm; Slit Width 4 nm
Column: Daicel CHIRALPAK IA-3, 4.6×100 mm, 3 μm (Daicel Part #80523)
Mobile Phase: 0.1% Trifluoroacetic acid (TFA, HPLC grade) in Methyl tent-butyl ether (MTBE, HPLC grade)
Injection Vol: 10 μL (include needle wash with diluent after each injection)
Flow Rate: 2.0 mL/min
Temperature: Column =35° C./Autosampler =Ambient
Run Time: 10 minutes
Retention time: ˜2.7 minutes (Atropisomer 1) and ˜4.2 minutes (Atropisomer 2).
   The following HPLC Sequence was used:

| Step | Sample | No. of Injections |
|---|---|---|
| 1 | Diluent | ≧2 |
| 2 | PLOQ Standard | 1 |
| 3 | Reference Standard | 1 |
| 4 | Diluent | 2 |
| 5 | Sample 1 | 1 |
| 6 | Sample 2 | 1 |
| 7 | Sample 3 | 1 |

### Step 5: Calculation

The enantiomeric purity and excess of atropisomers of Compound (I) was determined by % peak area responses of the isomers in the sample preparation, as described below.

### Enantiomeric Purity

Use the peak area responses to determine the enantiomeric purity of the Compound (I) atropisomers, according to the following equation:

$$Enantiomeric Purity = \frac{Atropisomer 1 peak area}{\begin{matrix}Atropisomer 1 peak area + \\ Atropisomer 2 peak area\end{matrix}} * 100$$

### Enantiomeric Excess

The peak area responses was used to determine the enantiomeric excess of the Compound (I) atropisomers, according to the following equation:

Enantiomeric Excess=(Atropisomer 1% peak area−Atropisomer 2% peak area)

### Step 6: Results

The following atropisomers were obtained: Atropisomer 1 Retention time: ˜2.7 minutes and Atropisomer 2 Retention time: ˜4.2 minutes. Sample chromatograms are presented in FIG. 1A and FIG. 1B.

### Example 3

### Isolation of Pure Atropisomers—Preparative Scale Chiral Separation

Compound (I) (2.5 g) was chromatographed on a SemiPrep HPLC unit using a 3 cm id ×25 cm L column, under the operating conditions below, to provide the individual atropisomers.
Chiral stationary phase: CHIRALPAK® IA 5 μm
   MTBE/DCM/TFA
Mobile phase:
   80/20/0.1
Flow rate: 40 mL/min
Temperature: Ambient
UV detection: 320 nm
Feed concentration 3.33 g/L (mobile phase)
Injection volume 25 mL every 6.5 min

The feed solution was filtered before injection onto the column. The collected fractions were evaporated to dryness (40° C.), and the final products dried overnight (vacuum oven, 35° C.) to provide the separate atropisomers as colorless glassy solids, containing residual amounts of MTBE and TFA.

| | Atropisomer 1 | Atropisomer 2 |
|---|---|---|
| Weight (g) | 1.40 | 1.55 |
| Recovery* | 112% | 124% |
| Enantiomeric purity % e.e. | 98.4 | 99.5 |

*High yields due to the presence of residual TFA.

### Example 4

### Optical Rotation

Optical activity is the ability to rotate a beam of plane-polarized light. Polarimetry is the measurement of optical activity. The optical rotation of the two separate atropisomers of Compound (I) was determined as follows: Atropisomer 1 (20.858 mg) was dissolved in methanol (2.0 mL) to provide a 0.010429 g/mL solution. Atropisomer 2 (20.580 mg) was dissolved in methanol (2.0 mL) to provide a 1.0290 g/100mL solution. The optical rotation of each atropisomer was measured on a Perkin-Elmer Polarimeter 341, under the following conditions:
Wavelength: 589 nm (Na)
Cell path: 100.00 mm
Temperature: 25° C.
Integration time: 50.0 (five runs)
   The specific rotation is calculated according to:

$$specific rotation = \frac{observed rotation}{path length \times concentration}$$

where the concentration of the solution is in g/mL and cell path length is in decimeters.

The results are shown in the table 2 below.

TABLE 2

| | |
|---|---|
| Atropisomer 1 | Atropisomer 1 |

| | | Specific | | Specific |
|---|---|---|---|---|
| Time | Rotation | Rotation | Rotation | Rotation |
| 50 s | −0.096 | −9.181 | +0.079 | +7.717 |
| 100 s | −0.093 | −8.917 | +0.079 | +7.717 |
| 150 s | −0.096 | −9.215 | +0.080 | +7.732 |
| 200 s | −0.098 | −9.391 | +0.082 | +7.937 |
| 250 s | −0.092 | −8.795 | +0.080 | +7.818 |
| | Average = | −9.099 deg | Average = | +7.784 deg |

Thus, Atropisomer 1 is (−) or levorotatory; that is, it rotates linearly polarized light to the left (counterclockwise) by 9.099 degrees and Atropisomer 2 is (+) or dextrorotatory; that is, it rotates linearly polarized light to the right (clockwise) by 7.784 degrees.

### Example 5

### Atropisomer Interconversion

The individual atropisomers of Compound (I), atropisomer 1 and atropisomer 2 (elution order from normal-phase chiral chromatography) were exposed to various conditions, including thermal challenge in both organic and aqueous media and exposure to simulated biological conditions, to ascertain whether they readily interconvert.

### Example 5A

### Thermal Challenge in Organic Solvent (Acetonitrile, 60° C., 4 Days)

Atropisomer 1 was dissolved in acetonitrile to a concentration of 0.25 mg/mL. Atropisomer 2 was dissolved in acetonitrile to a concentration of 0.25 mg/mL.

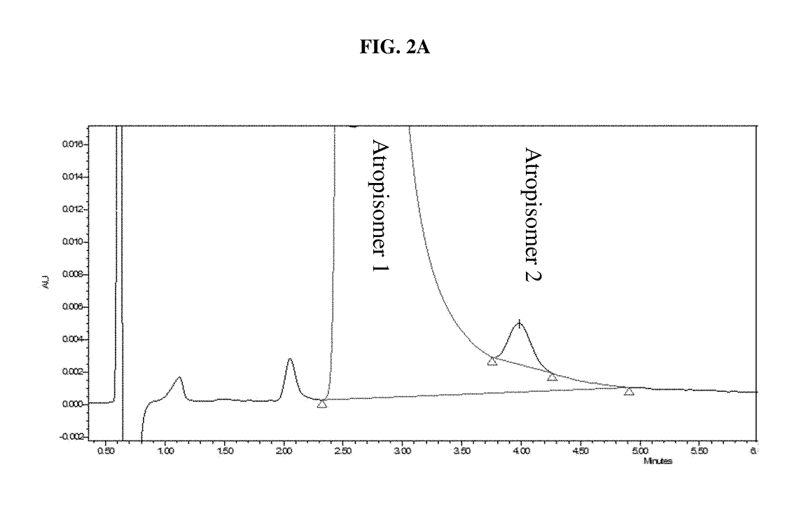

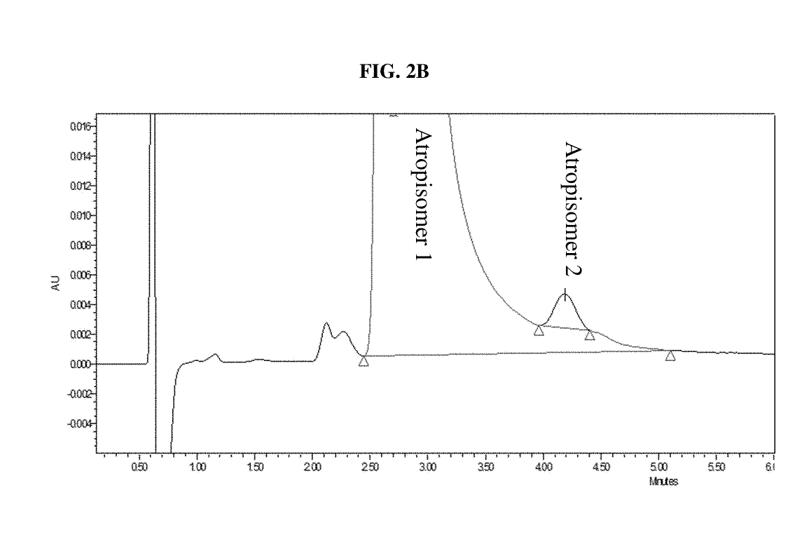

Samples of each separate atropisomers were were held at 60° C. for four days and then analyzed by high performance liquid chromatography (AGMS02 Agilent 1100) on a normal phase amylose-based chiral column (ChiralPak IA-3, 3 μm, 100×4.6 mm, column 244), using isocratic elution and UV detection at 226 nm (see example 1). Table 3 below shows the amounts of each atropisomer indicating no interconversion was observed. Chromatograms of Atropisomer 1 at time=0 and after heating at 60° C. for four days are shown in FIG. 2A and FIG. 2B respectively.

TABLE 3

| Sample | % Atropisomer 1 | % Atropisomer 2 |
|---|---|---|
| Atropisomer 1, t₀ | 99.7 | 0.3 |
| Atropisomer 2, t₀ | 1.0 | 99.0 |
| Atropisomer 1, 60° C. 4 days | 99.7 | 0.3 |
| Atropisomer 2, 60° C. 4 days | 1.0 | 99.0 |

### Example 5B

### Thermal Challenge in Aqueous Solvent (60° C., 100° C., 4 Days)

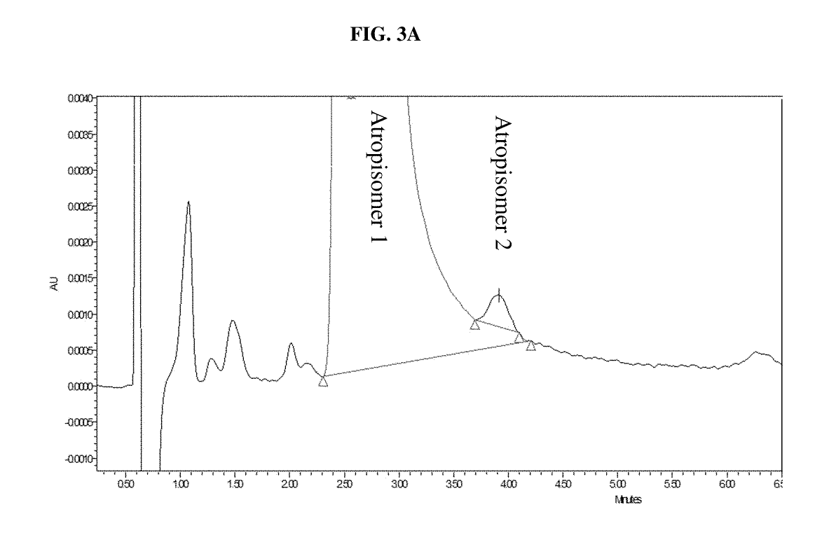

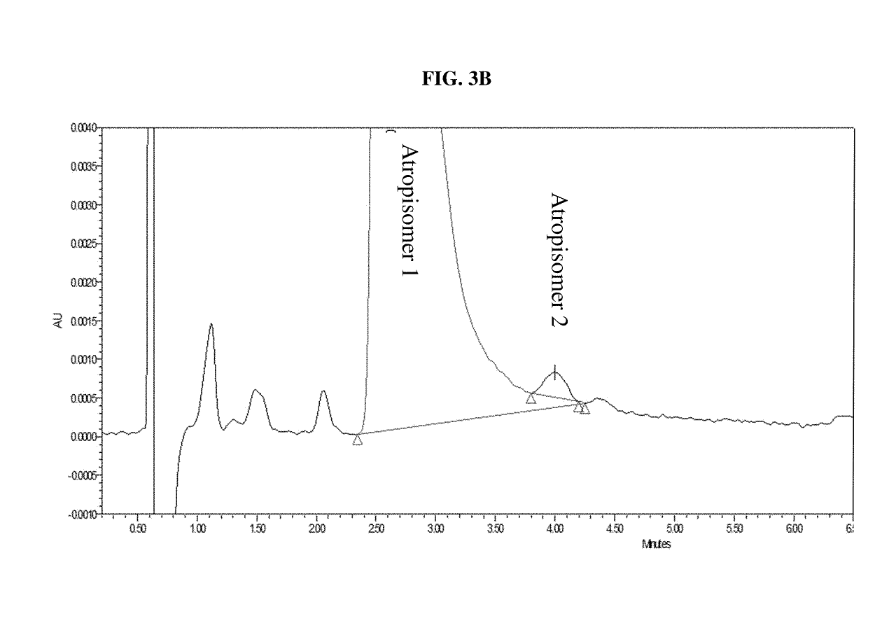

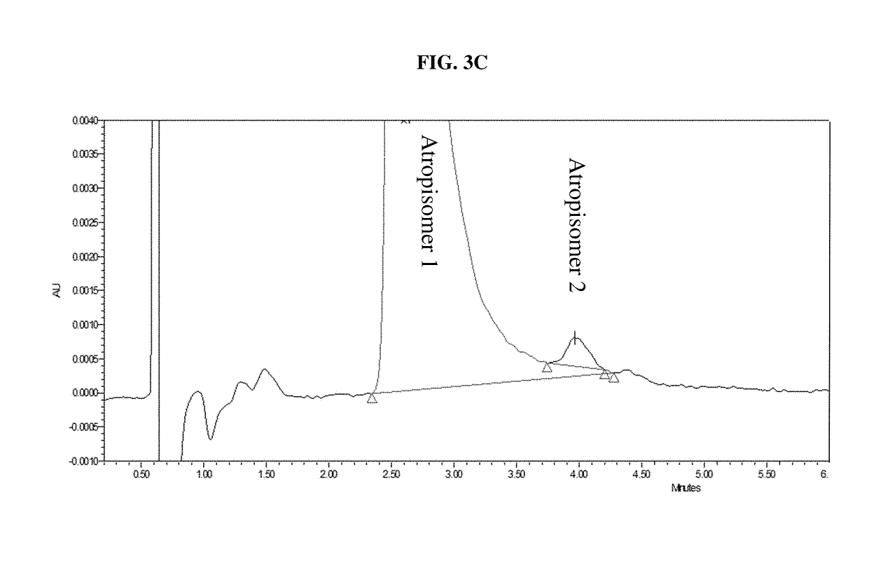

Atropisomer 1 was dissolved in dilute aqueous sodium bicarbonate solution. Atropisomer 2 was dissolved in dilute aqueous sodium bicarbonate solution. The solubility of Compound (I) in water is low; so to achieve aqueous dissolution the compounds were dissolved in dilute sodium bicarbonate solution. Samples of the resulting solutions were exposed to room temperature (rt), 60° C., and 100° C. for four days. After four days, the samples were concentrated to dryness (SpeedVac evaporator—DNA120, property H001630), redissolved in acetonitrile and analyzed by high performance liquid chromatography (AGMS02 Agilent 1100) on a normal phase amylose-based chiral column (ChiralPak IA-3, 3 μm, 100×4.6 mm, column 244), using isocratic elution and UV detection at 226 nm (see example 1). Table 4 below shows the amounts of each atropisomer observed. Sample chromatograms at t_{0,} 60° C., and 100° C. after four days, are depicted in FIG. 3A, FIG. 3B and FIG. 3C respectively.

TABLE 4

| Sample | % Atropisomer 1 | % Atropisomer 2 |
|---|---|---|
| Atropisomer 1, t₀ (control) | 99.8 | 0.2 |
| Atropisomer 2, t₀ (control) | 0.4 | 99.6 |
| Atropisomer 1, rt, 4 days | 99.8 | 0.2 |
| Atropisomer 2, rt, 4 days | 0.4 | 99.6 |
| Atropisomer 1, 60° C. 4 days | 99.8 | 0.2 |
| Atropisomer 2, 60° C. 4 days | 0.4 | 99.6 |
| Atropisomer 1, 100° C. 4 days | 99.6 | 0.4 |
| Atropisomer 2, 100° C. 4 days | 0.6 | 99.4 |

No interconversion was observed for samples held at room temperature or 60° C. 0.2% interconversion was observed for samples held at 100° C., after four days.

### Example 5C

### Simulated Biological Conditions—Tris Buffer

Atropisomer 1 was dissolved in DMSO to concentrations of 40 mM, 4.0 mM, or 0.4 mM. Atropisomer 2 was dissolved in DMSO to concentrations of 40 mM, 4.0 mM, or 0.4 mM.

2 μM of each atropisomer solution was mixed with Tris buffer (1998 μL, 50 mM, pH 7.4) to make 40 μM, 4.0 μM, or 0.4 μM incubation solutions, respectively, and incubated at 37° C. Aliquots (100 μL) of each incubation solution were removed immediately after mixing and at 0.5 hr, 1 hr, 4 hr, 8 hr and 24 hr timepoints, mixed with chilled acetonitrile (300 μL), transferred to 1.5-mL centrifuge tubes and concentrated to dryness (DNA120 speedvac , high setting). Acetonitrile (100 μL) was added, the tube sonicated (5 minutes) and centrifuged (2 minutes), and the resulting supernatant transferred to vials for analysis by LC/MS (selective-ion monitoring mode, scanning for masses 404 and 406). No interconversion of atropisomers was observed.

### Example 5D

### Simulated Biological Conditions—Human Serum

Atropisomer 1 was dissolved in DMSO to concentrations of 40 mM, 4.0 mM, or 0.4 mM. Atropisomer 2 was dissolved in DMSO to concentrations of 40 mM, 4.0 mM, or 0.4 mM.

2 μM of each atropisomer solution was mixed with human serum (1998 μL) to make 40 μM, 4.0 μM, or 0.4 μM incubation solutions, respectively, and incubated at 37° C. Aliquots (100 μL) of each incubation solution were removed immediately after mixing and at 0.5hr, 1hr, 4hr, 8hr and 24hr and mixed with chilled acetonitrile (300 μL). Precipitated proteins were removed by centrifugation (3300×g, 15min, 4° C.). The supernatant fractions were transferred to 1.5-mL centrifuge tubes and concentrated to dryness (DNA120 speedvac , high setting). Acetonitrile (100 μL) was added, the tube sonicated (5 minutes) and centrifuged (2 minutes), and the resulting supernatant transferred to vials for analysis by LC/MS (selective-ion monitoring mode, scanning for masses 404 and 406). No interconversion of atropisomers was observed.

### Example 6

### Inhibition of URAT1

The inhibition potential of the individual atropisomers of Compound (I) was determined against the human URAT1 transporter, using a cell-based assay in HEK293 cells stably expressing URAT1.

Subcloning of Human Uric Acid Transporters—The full-length human URAT1 cDNA (SLC22A12) was subcloned from pCMV6-XL5 (Catalog #SC125624, OriGene Technologies, Inc. Rockville, Md.) into the pCMV6-neo vector (Origene) using NotI restriction sites in order to generate a eukaryotic expression plasmid containing a drug resistance selection marker, which was named pCMV6-neo URAT1. Gene sequencing confirmed the sequence of hURAT1 as outlined in Genbank (Accession #NM_144585.2).

Generation and Screening of HEK293 Stable Cell Lines—HEK293 human embryonic kidney cells (ATCC #CRL-1573, Manassas, Va.) were propagated in EMEM tissue culture medium as described by ATCC in an atmosphere of 5% CO₂ and 95% air. HEK293 cells were transfected with the expression plasmids described above (pCMV6-neo URAT1 or pCMV6-neo OAT4) using Lipofectamine 2000 (Invitrogen, Carlsbad, Calif.), according to the manufacturer's instructions. The lipid/DNA transfection mixture was removed after five hours on the cells and fresh growth medium was added. After another 24 hr the cells were split into 10 cm plates. The next day G418 (Gibco, Carlsbad, Calif.) selection agent was added to the growth medium at a final concentration of 0.5 mg/ml. Fresh G418-containing medium was added every three days until drug-resistant colonies were obtained and also when the majority of mock-transfected cells were dead. Drug-resistant colonies were isolated by cloning rings into 48-well plates and cultured until sufficient cell numbers were available for screening.

HEK293 hURAT 1 Stable Cell-Line Uric Acid Uptake Assay—Cells were seeded onto 96-well poly-D-lysine coated tissue culture plates at a density of 1.25×10⁵ cells per well and grown at 37° C. overnight. The next day the cell culture was washed once with Wash Buffer (125 mM sodium gluconate, 25 mM HEPES pH 7.4). Test compounds were diluted in AB buffer with 1.5 percent DMSO and preincubated with the cells for 5 minutes at room temperature in a volume of 30 μl. 15 μl of 500 μM¹⁴C-uric acid diluted in AB buffer was added to the plate and incubated for 10 minutes at room temperature. Free ¹⁴C-uric acid was removed by washing cells 4 times with Wash Buffer. Cells were lysed by adding 150 μl of MicroScint 20 scintillation fluid (PerkinElmer) to each well and radioactivity was counted the following day using a Beckman TopCount plate reader. The half maximal inhibitory concentration (IC₅₀) to inhibit URAT1 was measured by the decrease in C¹⁴-labeled uric acid uptake with increasing concentrations from 0 to 200 μM.

Test Compounds—Atropisomer 1 and atropisomer 2 were isolated by HPLC, as described herein. Atropisomer 1 Purity (by HPLC): 91.9% (% w/w) and Atropisomer 2 Purity (by HPLC): 88.2%(% w/w).

Calculation—The IC₅₀ was calculated using Sigmoidal Dose-Response Model using XLFIT(IDBS, Alameda, Calif.). Average (of four runs; each run in triplicate) IC₅₀ and standard error of the mean (SEM) for each atropisomer are shown in the table below. Atropisomer 2 demonstrates approximately 4 fold greater inhibition of URAT1 than atropisomer 1 as seen in table 5 below.

TABLE 5

| Atropisomer | URAT1 IC₅₀ | SEM |
|---|---|---|
| 50/50 Mixture | 9.5 | 1.3 |
| 1 | 17.4 | 3.2 |
| 2 | 4.3 | 1.1 |

### Example 7

### Inhibition of OAT4

The inhibition potential of the individual atropisomers of Compound (I) was assessed against the human OAT4 transporter, using cell-based assays in HEK293 cells, stably expressing the OAT4.

Subcloning of Human hOAT4 Transporters—The full-length human OAT4 cDNA (SLC22A11) was subcloned from pCMV-SPORT6 (Clone ID 5190509, Open Biosystems, Huntsville, Ala.) into pCMV6-neo. EcoRl and Hindlll digestions were performed to release the OAT4 cDNA from pCMV-SPORT6 and directionally clone it into pCMV6-neo, which was named pCMV6-neo OAT4. Gene sequencing confirmed the sequence of hOAT4 as outlined in Genbank (Accession #NM_018484).

Generation and Screening of HEK293 Stable Cell Lines—HEK293 human embryonic kidney cells (ATCC #CRL-1573, Manassas, Va.) were propagated in EMEM tissue culture medium as described by ATCC in an atmosphere of 5% CO2 and 95% air. HEK293 cells were transfected with the expression plasmids described above (pCMV6-neo URAT1 or pCMV6-neo OAT4) using Lipofectamine 2000 (Invitrogen, Carlsbad, Calif.), according to the manufacturer's instructions. The lipid/DNA transfection mixture was removed after five hours on the cells and fresh growth medium was added. After another 24 hr the cells were split into 10 cm plates. The next day G418 (Gibco, Carlsbad, Calif.) selection agent was added to the growth medium at a final concentration of 0.5 mg/ml. Fresh G418-containing medium was added every three days until drug-resistant colonies were obtained and also when the majority of mock-transfected cells were dead. Drug-resistant colonies were isolated by cloning rings into 48-well plates and cultured until sufficient cell numbers were available for screening.

HEK293 hOAT4 Stable Cell Line 6-carboxyflorescein Uptake Assay—Cells were seeded onto 96-well poly-D-lysine coated tissue culture plates at a density of 1.25×10⁵ cells per well and grown at 37° C. overnight. The next day cells were washed once with KRP buffer. Compounds diluted in KRP buffer with 1.5 percent DMSO were preincubated with the cells for 5 minutes at room temperature in a volume of 30 μl. 15 μl of 75 μM 6-Carboxyflorescein diluted in KRP buffer was added to the plate and incubated for 10 minutes at room temperature. Free 6-Carboxyflorescein was removed by washing cells 4 times with Wash Buffer. Cells were lysed by adding 150 μl of 1 N NaOH to each well and incubating at 37° C. for 1 hour. Plates were then read on the Molecular Devices Analyst HT plate reader. The half maximal inhibitory concentration IC₅₀ to inhibit OAT4 was measured by the decrease in 6-carboxyflorescein uptake with increasing concentrations from 0 to 200 μM.

Test Compounds—Atropisomer 1 and atropisomer 2 were isolated by HPLC, as described herein. Atropisomer 1 Purity (by HPLC): 91.9% (% w/w) and Atropisomer 2 Purity (by HPLC): 88.2% (% w/w).

Calculation—The IC₅₀ was calculated using Sigmoidal Dose-Response Model using XLFIT(IDBS, Alameda, Calif.). Average (of six runs; each run in triplicate) IC₅₀ and standard error of the mean (SEM) for each atropisomer are shown in the table 6 below. Atropisomer 1 and atropisomer 2 are equally active against OAT4.

TABLE 6

| Atropisomer | OAT4 IC₅₀ | SEM |
|---|---|---|
| 50/50 Mixture | 4.25 | 0.51 |
| 1 | 3.68 | 0.27 |
| 2 | 4.36 | 0.4 |

### Example 8

### In Vitro Metabolism

Previous in vitro metabolism studies (human pooled liver microsomes (HLM) and hepatocytes) with racemic Compound (I) (2-((5-bromo-4-(4-cyclopropylnaphthalen-1-yl)-4H-1,2,4-triazol-3-yl)thio)acetic acid) have shown the formation of 3 oxidative metabolites: M3 (hydroxylated); M4 (dihydrodiol—formed via epoxide intermediate M3c subsequently hydrolyzed by microsomal epoxide hydrolase (mEH); and M6 (S-dealkylated) as seen in scheme 2 below.

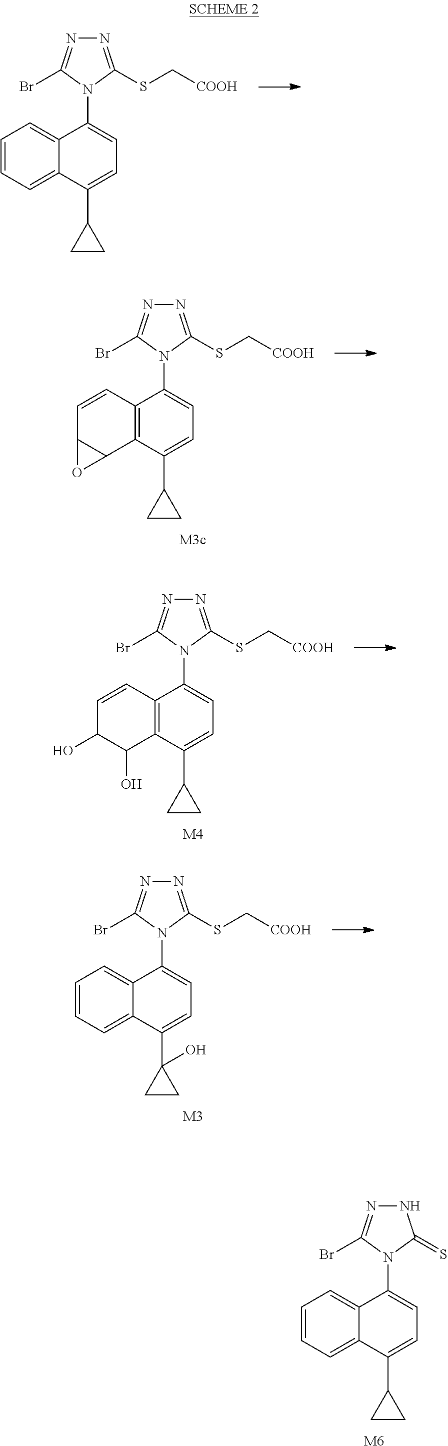

The stereoselective metabolism of the two separate atropisomers—atropisomer 1 and atropisomer 2, was evaluated as follow.

### Equipment

Autosampler: CTC Analytics PAL System, Leap Technologies

Balance: Mettler Toledo AX205, Mettler-Toledo AG, Laboratory & Weighing Technologies

Balance: Explorer Model E00640, Ohaus Corporation

Centrifuge, Allegra® X-15R, Beckman Coulter

Liquid chromatography binary pump: series 1100, Agilent Technologies

Liquid chromatography degasser: series 1100, Agilent Technologies

Mass spectrometer: API4000 triple quadrupole LC-MS/MS system, Applied Biosystems Inc/MDS Sciex

Mass spectrometer data software: Analyst 1.5.1, Applied Biosystems Inc/MDS Sciex

Water bath: Shaker Bath Model 25, Precision

Column: Luna 5 μm C8 (2), 4.6×150 mm, catalog number 00F-4249-E0, Phenomenex

### Reagents

Acetonitrile: HPLC grade, (Fisher Scientific, catalog A998-4)

Acetonitrile: LC/MS grade, (Fisher Scientific, catalog A955-4) 0.1% formic acid in acetonitrile: Burdick & Jackson HPLC spectrophotometry grade, catalog number 441-4, Honeywell International Inc.

Dimethyl sulfoxide (DMSO): Fisher Scientific, catalog BP231

7-Hydroxycoumarin: Aldrich, catalog H24003-10G

Formic acid: Fisher Scientific catalog A118-P-100

Magnesium chloride (MgCl₂) solution (1M): Sigma-Aldrich, catalog M1028

NADPH (β-Nicotinamide adenine dinucleotide phosphate, reduced form): Sigma-Aldrich, catalog N7505

Potassium phosphate buffer powder: Sigma-Aldrich, catalog P7994

Valpromide: catalog number V3640-10 mg, Sigma-Aldrich

Water: HPLC grade, EMD Millipore catalog WX0008-1

0.1% formic acid in water: Burdick & Jackson HPLC spectrophotometry grade, catalog 452-4, Honeywell International Inc.

### Prepared Solutions

Mobile phase solution A: 0.1% formic acid in water: formic acid (4.545 mL) was added to water (4 L) and shaken by hand to mix.

Mobile phase solution B: 0.1% formic acid in acetonitrile: formic acid (2.27 mL) was added to acetonitrile (2 L) and shaken by hand to mix.

Phosphate buffer solution: 200 mM potassium phosphate buffer, pH=7.4: phosphate buffer powder (1 package) was added to water (approximately 1.8 L), mixed well and then total volume brought to 2.0 L with water.

Internal standard solution: 7-hydroxycoumarin in acetonitrile (0.1 μM): 7-hydroxycoumarin (2.8 mg) was dissolved in acetonitrile (3.453 mL) to make 5 mM stock solution. 10 μL of 5 mM solution were added to 500 mL of acetonitrile to make 0.1 μM of 7-hydroxycoumarin solution.

### Test Substances

Atropisomer 1 Purity (by HPLC): 91.9% (% w/w) and Atropisomer 2 Purity (by HPLC): 88.2% (% w/w).

### Incubation Procedures

### Incubations With Human Liver Microsomes

Human liver microsomes (0.5 mg/mL; mixed gender, ultrapooled, BD Biosciences, catalog number 452117) were incubated at 37±1° C. in 0.2 mL (final volume) of incubation mixtures containing potassium phosphate buffer (100 mM, pH7.4), MgCl₂ (3 mM), NADPH (1 mM), and the atropisomers (1, 10, or 50 μM) (all experiments performed in duplicate). Stock solutions of atropisomers were prepared with DMSO at 100 mM. The final concentration of DMSO in the incubation was at or less than 0.05%. Reactions were initiated by addition of NADPH solution and terminated at predetermined time point (30 min) by addition of stop reagent (300 μL chilled acetonitrile containing 0.1 μM of 7-hydroxycoumarin as an internal standard). After incubation, microsomal proteins were precipitated by centrifugation at 3300×g for 15 min at 4° C. Supernatant fractions were analyzed by LC-MS/MS.

### Incubation With Liver Microsomes in the Presence of mEH Inhibitor Valpromide

Human liver microsomes (0.5 mg/mL; mixed gender, ultrapooled, BD Biosciences, catalog number 452117) were incubated at 37±1° C. in 0.2 mL (final volume) of incubation mixtures containing potassium phosphate buffer (100 mM, pH7.4), MgCl₂ (3 mM), NADPH (1 mM), atropisomer (1, 10, or 50 μM) and valpromide (0, 50, or 100 μM) (all experiments performed in duplicate). Reactions were initiated by addition of NADPH solution and terminated at predetermined time point (30 min) by addition of stop reagent (300 μL chilled acetonitrile containing 0.1 μM of 7-hydroxycoumarin as an internal standard). After incubation, microsomal proteins were precipitated by centrifugation at 3300×g for 15 min at 4° C. Supernatant fractions were analyzed by LC-MS/MS.

### LC-MS/MS Method

HPLC mass spectrometer system: An API4000 triple quadrupole mass spectrometer equipped with electrospray ionization ion source was used for quantitation and operated in positive mode. The HPLC system consisted of a CTC Analytics PAL autosampler, an Agilent 1100 series degasser (G1379A), and an Agilent 1100 series binary pump (G1312A). Separation of atropisomers and metabolites was achieved by a reversed phase C8 column (Phenomenex, Luna C8(2), 150×4.6 mm) without chiral separation. HPLC conditions were as follows:
Mobile Phase: A: 0.1% formic acid in water
   B: 0.1% formic acid in acetonitrile
Flow Rate: 0.9 mL/min
Injection Volume: 10.0 μL
Gradient Profile:

| Time | A (%) | B (%) |
|---|---|---|
| 0 | 90 | 10 |
| 1.0 | 90 | 10 |
| 10.0 | 10 | 90 |
| 15.9 | 10 | 90 |
| 16.0 | 90 | 10 |
| 20 | 90 | 10 |

API4000 triple quadrupole system mass spectrometric conditions:

| | MS/MS Ion Transition | Declustering | Collision |
|---|---|---|---|
| Analytes | (m/z, amu) | Potential | Energy |
| Compound (I) | 404 → 386 | 86 | 29 |
| M3, M3c | 420.2 → 402 | 86 | 30 |
| M4 | 438 → 402 | 86 | 30 |
| M6 | 346 → 152.3 | 86 | 55 |
| 7-Hydroxycoumarin | 163 → 107 | 30 | 30 |
| (IS) | | | |

The formation of M3, M3c, M4 and M6 from each atropisomer in liver microsomal and recombinant CYP incubations was determined, based on the peak area ratio of analyte over internal standard (IS)

### Human Liver Microsomes

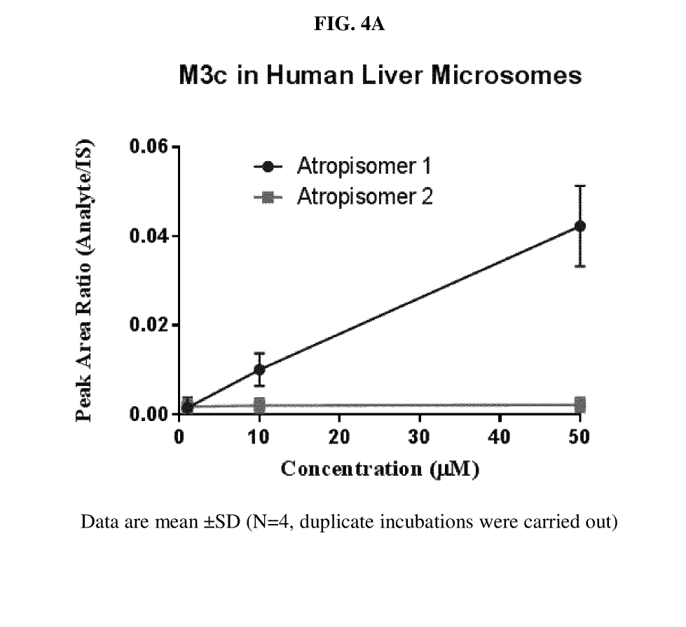

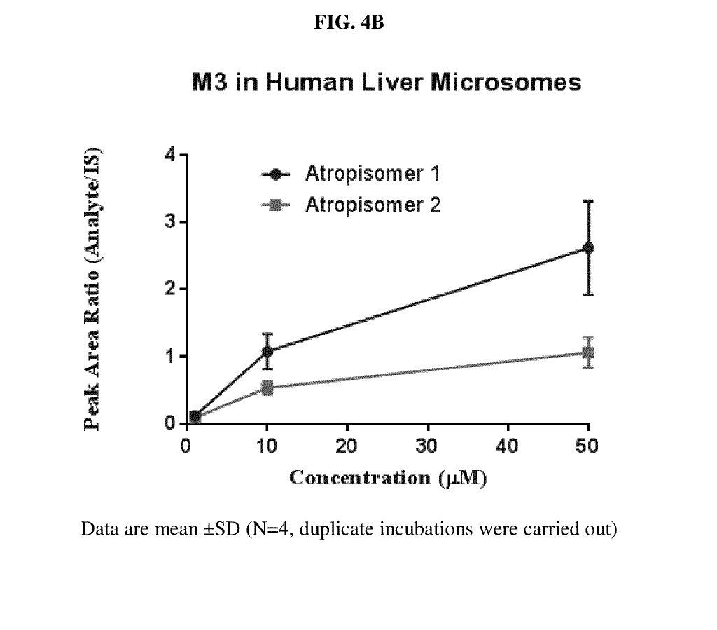

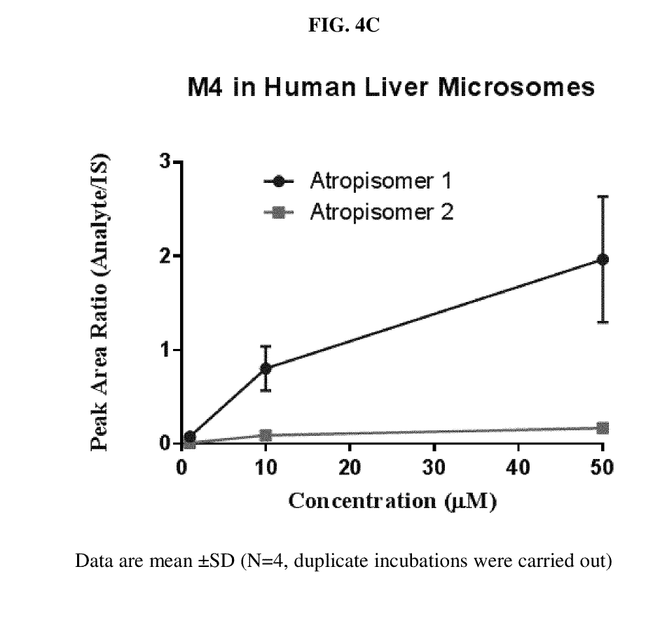

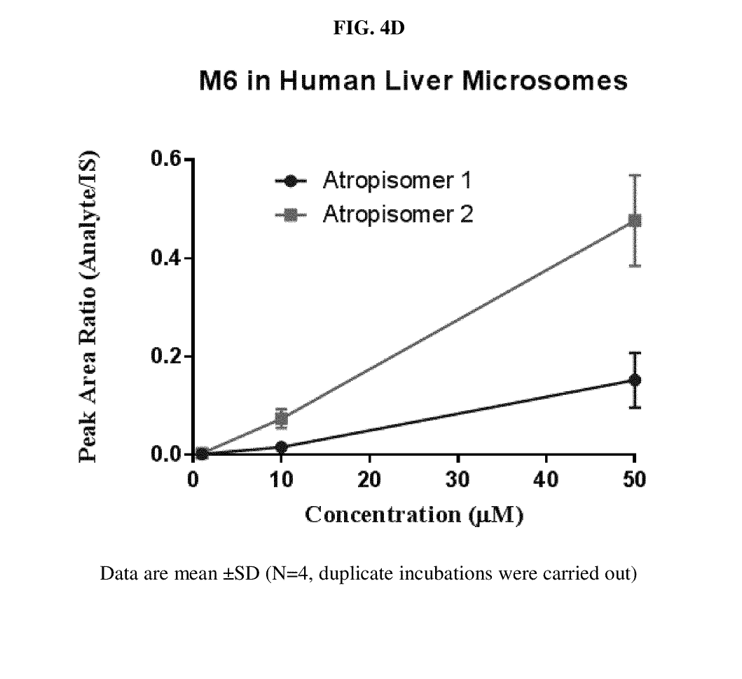

Table 7 below and FIG. 4A, FIG. 4B, FIG. 4C, and FIG. 4D, show the ratio of metabolite formed (M3, M3c, M4 and M6) from Atropisomer 1 and Atropisomer 2 in human liver microsomes indicating that the metabolism of Compound (I) atropisomers in human liver microsomes is stereoselective.

TABLE 7

| | Atropisomer | Metabolite Ratio |
|---|---|---|
| Metabolite | Concentration (μM) | Atropisomer 1:Atropisomer 2 |
| M3c | 1 | 1.43 |
| | 10 | 3.92 |
| | 50 | 16.1 |
| M4 | 1 | 7.68 |
| | 10 | 9.26 |
| | 50 | 11.8 |
| M3 | 1 | 1.33 |
| | 10 | 2.04 |
| | 50 | 2.48 |
| M6 | 1 | 0.552 |
| | 10 | 0.252 |
| | 50 | 0.317 |

M3c was predominantly formed from atropisomer 1; while atropisomer 2 produced only trace levels. M4 formation was 8-12× greater from atropisomer 1 than from atropisomer 2. M3 formation was 2× greater from atropisomer 1 as from atropisomer 2. M6 formation was ˜3× greater from atropisomer 2 than from to atropisomer 1.

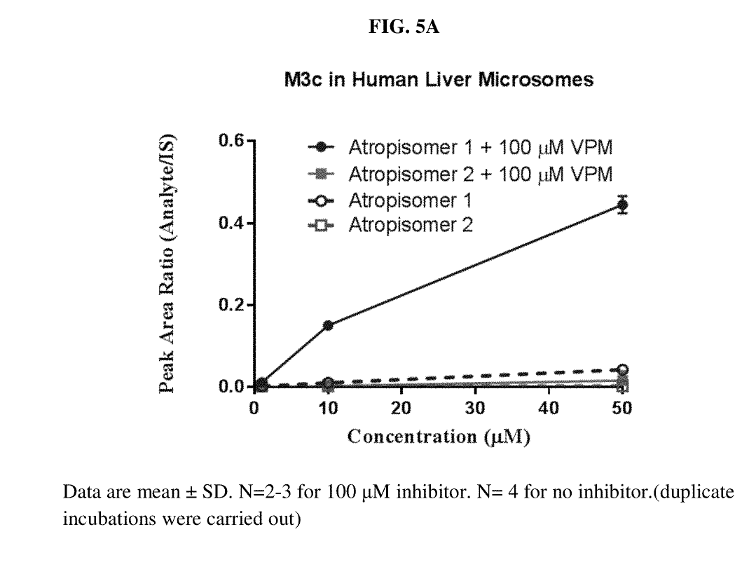

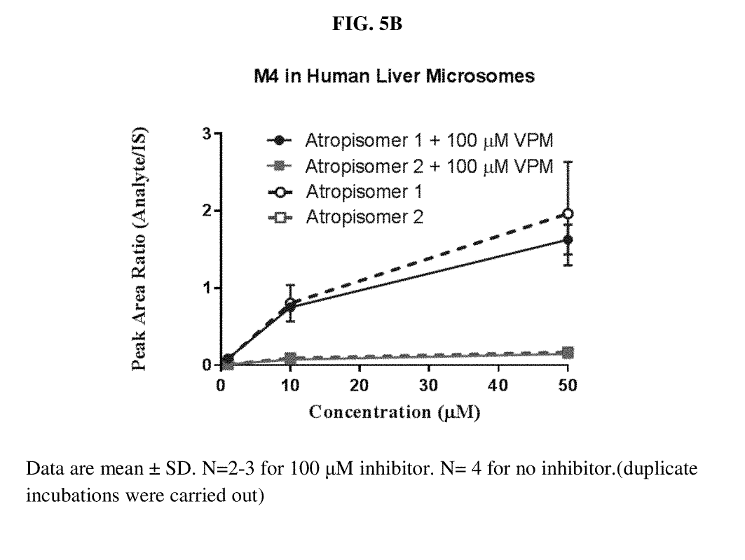

Table 8 below and FIG. 5A and FIG. 5B: show the ratio of metabolite formed (M3, M3c, M4 and M6) from Atropisomer 1 and Atropisomer 2 in human liver microsomes in the presence of mEH Inhibitor Valpromide (100 μM).

TABLE 8

Metabolite Ratio (1:2)

| | Atropisomer | | Valpromide |
|---|---|---|---|
| Metabolite | Conc (μM) | No Valpromide | (100 μM) |
| M3c | 1 | 1.43 | 37.0 |
| | 10 | 3.92 | 36.3 |
| | 50 | 16.1 | 47.6 |
| M4 | 1 | 7.68 | 7.76 |
| | 10 | 9.26 | 11.1 |
| | 50 | 11.8 | 12.1 |

M3c level with atropisomer 1 was greatly increased in the presence of valpromide (mEH inhibitor). M4 formation from atropisomer 1 was inhibited by approximately 14% to 29%. Formation of M3c CYP2C9 mediated was significantly greater from atropisomer 1 than atropisomer 2. This confirms that the mechanism of M4 formation from M3c, via epoxide hydrolysis is mediated by mEH. Stereoselective metabolism of atropisomers to M3c was confirmed with recombinant human CYP2C9 (See table 9). With CYP2C9 formation of M3c metabolite from atropisomer 1, and subsequent formation of M4 in liver microsomes of human was observed. In humans, no metabolites were observed in plasma at levels greater than 10% of the total exposure. Therefore, any potential impact of varying preferential metabolism of either atropisomer is not considered to be significant.

TABLE 9
Inhibitory Effect of Valpromide on M4 Formation in Human
Microsomal Incubations With Atropisomers (50 μM)

| Atropisomer | Valpromide Conc | % Inhibition |
|---|---|---|
| 1 | 0 μM | 0 |
| | 50 μM | 14.1 |
| | 100 μM | 29.1 |
| 2 | 0 μM | 0 |
| | 50 μM | 23.4 |
| | 100 μM | 30.8 |

N = 2; duplicate incubations were carried out in each experiment

### Example 9

### In Vivo Human Metabolism

11 adult male subjects were orally administered a single 400 mg dose of Compound (I) (racemic mixture). Plasma samples were collected prior to dosing (within 30 minutes before dosing) and at the following timepoints: 0.5, 1, 1.5, 2, 3, 4, 5, 6, 8, 10, 12, and 24 hours postdose. Urine was also collected. For quantitative determination, human plasma samples containing K3EDTA as anticoagulant were extracted by protein precipitation with acetonitrile containing deuterated Compound (I) as internal standard. The supernatant was diluted with injection solvent and analyzed by HPLC with tandem mass spectrometry (LC/MS/MS). An API 5000 triple quadrupole mass spectrometer, operated in positive TurbolonSpray® mode, was used to monitor the precursor→product ion transitions of m/z 404→106 and m/z 410→392 for Compound (I) and deuterated Compound (I), respectively. The calibration curves were linear over the concentration range between 5.00 ng/mL and 2000 ng/mL with a lower limit of quantification (LLOQ) of 5.00 ng/mL. The mean plasma concentrations (ug/mL) of Atropisomer 1, Atropisomer 2 and Compound (I) at the designated timepoints are presented in table 10 below.

TABLE 10

| | | | Racemic | |
|---|---|---|---|---|
| Time (hr) | Atropisomer 1 | Atropisomer 2 | mixture | Sum 1 + 2 |
| 0 | 0 | 0 | 0 | 0 |
| 0.5 | 0.065 | 0.108 | 0.157 | 0.173 |
| 1 | 1.725 | 2.573 | 3.900 | 4.298 |
| 1.5 | 3.597 | 4.975 | 7.860 | 8.572 |
| 2 | 4.455 | 5.765 | 9.380 | 10.220 |
| 3 | 5.744 | 6.921 | 11.400 | 12.665 |
| 4 | 4.563 | 5.577 | 9.080 | 10.140 |
| 5 | 3.428 | 4.314 | 6.980 | 7.742 |
| 6 | 1.960 | 2.322 | 3.810 | 4.283 |
| 8 | 0.811 | 0.918 | 1.530 | 1.729 |
| 10 | 0.415 | 0.495 | 0.815 | 0.910 |
| 12 | 0.248 | 0.309 | 0.503 | 0.557 |
| 24 | 0.033 | 0.063 | 0.084 | 0.096 |

Using the above data, the following plasma pharmacokinetic parameters were calculated using validated Phoenix WinNonlin, version 6.3 (Pharsight Corporation, Mountain View, Calif.) and are shown (geometric mean 95% CI) in tables 11, 12, and 13 below.

TABLE 11

| Analyte | Cₘₐₓ | Tₘₐₓ | AUC₀₋₂₄ | C_{24 h} | t_{1/2} |
|---|---|---|---|---|---|
| Atropisomer 1 | 6.23 | 3.00 | 25.9 | 0.0216 | 3.59 |
| Atropisomer 2 | 7.92 | 3.00 | 32.7 | 0.0508 | 4.67 |
| Racemic mixture | 12.8 | 3.00 | 52.9 | | 4.18 |

TABLE 12
Plasma Concentration (Mean)

| Analyte | Cₘₐₓ | AUC₀₋₂₄ |
|---|---|---|
| Atropisomer 1 | 6.6 | 27.1 |
| Atropisomer 2 | 8.2 | 33.8 |
| Racemic mixture | 13.5 | 54.9 |
| Sum 1 + 2 | 14.8 | 60.9 |

TABLE 13
Urine PK Parameters (Geometric mean, CI 95%)

| Analyte | Ae₀₋₂₄ (mg) | CL_{R0-24} (mL/min) |
|---|---|---|
| Atropisomer 1 | 66.3 | 42.6 |
| Atropisomer 2 | 101 | 51.6 |
| A1/A2 Ratio | 0.655 | 0.826 |

Cₘₐₓ: Maximum observed concentration (μg/mL)
Tₘₐₓ: Time of occurrence of maximum observed concentration (hr)
AUC₀-₂₄: Area under plasma concentration-vs-time curve, from time 0 to 24 hours postdose (μg·hr/mL)
C₂₄: Concentration 24 hours post dose (μg/mL)
t_{1/2}: Apparent terminal half-life (hr)

While certain embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments described herein are, in some circumstances, employed in practicing the invention. It is intended that the following claims define the scope of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

1. (+)-2-((5-bromo-4-(4-cyclopropylnaphthalen-1-yl)-4H-1,2,4-triazol-3-yl)thio)acetic acid.

2. (−)-2-((5-bromo-4-(4-cyclopropylnaphthalen-1-yl)-4H-1,2,4-triazol-3-yl)thio)acetic acid.

3-6. (canceled)

7. A pharmaceutical composition comprising either:
i. (+)-2-((5-bromo-4-(4-cyclopropylnaphthalen-1-yl)-4H-1,2,4-triazol-3-yl)thio)acetic acid, or a pharmaceutically acceptable salt thereof; or
ii. (−)-2-((5-bromo-4-(4-cyclopropylnaphthalen-1-yl)-4H-1,2,4-triazol-3-yl)thio)acetic acid, or a pharmaceutically acceptable salt thereof; or
iii. a mixture enriched in one atropisomer of 2-((5-bromo-4-(4-cyclopropylnaphthalen-1-yl)-4H-1,2,4-triazol-3-yl(thio)acetic acid; or a pharmaceutically acceptable salt thereof; and
a pharmacologically acceptable carrier, diluent, or excipient.

8. The pharmaceutical composition of claim 7, further comprising:
i. allopurinol; or
ii. febuxostat; or
iii. colchicine; or
iv. any combination thereof.

9-10. (canceled)

11. A method of treating hyperuricemia associated with gout in a human;
comprising administering to the human a therapeutically effective amount of:
i. (+)-2-((5-bromo-4-(4-cyclopropylnaphthalen-1-yl)-4H-1,2,4-triazol-3-yl)thio)acetic acid, or a pharmaceutically acceptable salt thereof; or
ii. (−)-2-((5-bromo-4-(4-cyclopropylnaphthalen-1-yl)-4H-1,2,4-triazol-3-yl)thio)acetic acid, or a pharmaceutically acceptable salt thereof; or
iii. a mixture enriched in one atropisomer of 2-((5-bromo-4-(4-cyclopropylnaphthalen-1-yl)-4H-1,2,4-triazol-3-yl)thio)acetic acid, or a pharmaceutically acceptable salt thereof.

12. The method of claim 11 comprising administering to the human a therapeutically effective amount of (+)-2((5-bromo-4-(4-cyclopropylnaphthalen-1-yl)-4H-1,2,4-triazol-3-yl)thio)acetic acid, or a pharmaceutically acceptable salt thereof.

13. The method of claim 11 comprising administering to the human a therapeutically effective amount of mixture enriched in (+)-2-((5-bromo-4-(4-cyclopropylnaphthalen-1-yl)-4H-1,2,4-triazol-3-yl)thio)acetic acid, or a pharmaceutically acceptable salt thereof.

14. The method of claim 11 comprising administering to the human a therapeutically effective amount of (−)-2-((5-bromo-4-(4-cyclopropylnaphthalen-1-yl)-4H-1,2,4-triazol-3-yl)thio)acetic acid, or a pharmaceutically acceptable salt thereof.

15. The method of claim 11 comprising administering to the human a therapeutically effective amount of mixture enriched in (−)-2-((5-bromo-4-(4-cyclopropylnaphthalen-1-yl)-4H-1,2,4-triazol-3-yl)thio)acetic acid, or a pharmaceutically acceptable salt thereof.

16. The method of claim 11 further comprising administering:
i. allopurinol; or
ii. febuxostat; or
iii. colchicine; or
iv. any combination thereof.

17. (+)-2-((5-bromo-4-(4-cyclopropylnaphthalen-1-yl)-4H-1,2,4-triazol-3-yl)thio)acetic acid of claim 1, wherein the (+)-2((5-bromo-4-(4-cyclopropylnaphthalen-1-yl)-4H-1,2,4-triazol-3-yl)thio)acetic acid is provided in at least 75% enantiomeric excess.

18. (−)-2-((5-bromo-4-(4-cyclopropylnaphthalen-1-yl)-4H-1,2,4-triazol-3-yl)thio)acetic acid of claim 2, wherein the (−)-2-((5-bromo-4-(4-cyclopropylnaphthalen-1-yl)-4H-1,2,4-triazol-3-yl)thio)acetic acid is provided in at least 75% enantiomeric excess.
